(19) United States
(12) Patent Application Publication
(10) Pub. No.: US 2025/0049361 A1
(43) Pub. Date: Feb. 13, 2025

(54) Equipment Kit and Container With Tools for Performing Blood Draw Procedures and Methods of Use

(71) Applicant: Becton, Dickinson and Company, Franklin Lakes, NJ (US)

(72) Inventors: Craig Owen Russ, Wayne, NJ (US); Ashay Arvind Alur, Mesa, AZ (US); Edward Goldberg, Rochester, NY (US); Bart D. Peterson, Farmington, UT (US); Bradley M. Wilkinson, North Haledon, NJ (US); Martin K. Matejka, Columbus, NE (US); C. Mark Newby, Kamas, UT (US)

(21) Appl. No.: 18/721,935

(22) PCT Filed: Jan. 3, 2023
(86) PCT No.: PCT/US2023/010047

§ 371 Date: Jun. 20, 2024

Related U.S. Application Data
(60) Provisional application No. 63/296,319, filed on Jan. 4, 2022.

(51) Int. Cl. A61B 5/15; A61B 5/153; A61M 5/00

(52) U.S. Cl. CPC A61B 5/150305; A61B 5/150328; A61B 5/150343; A61B 5/150992; A61B 5/153; A61M 5/002; A61M 5/008

(57) ABSTRACT

A container configured to store tools used by a healthcare worker when performing a blood draw and collection procedure includes a tray and packaging. The tray includes a plurality of chambers sized to receive the tools used by the healthcare worker for the blood draw and collection procedure. The tools include at least one disposable disinfecting device for disinfecting portions of the tools and at least one tube for storing a blood sample. The packing, when in a closed or prior-to-use position, is over openings of the plurality of chambers of the tray, thereby securely containing the tools within the chambers of the tray. In an open or ready-for-use position, the packaging is separated from the openings of the plurality of chambers of the tray so that the healthcare worker can access the tools contained within the chambers while performing the blood draw and collection procedure.

### CROSS-REFERENCE TO RELATED APPLICATION

The present application claims priority to U.S. Provisional Application No. 63/296,319, entitled “Equipment Kit and Container with Tools for Performing Blood Draw Procedures and Methods of Use”, filed Jan. 4, 2022, the entire disclosure of which is hereby incorporated by reference in its' entirety.

### BACKGROUND OF THE INVENTION

### Field of the Invention

The present disclosure relates generally to packaging, containers, boxes, and kits that contain tools used for medical procedures. More particularly, the present disclosure relates to an equipment kit and/or container containing most or all tools and associated items needed for performing a medical procedure, in particular, a blood draw and collection procedure.

### Description of Related Art

Blood collection procedures, such as blood collection from a catheter or central line of a patient, rely heavily on the skill and training of the healthcare worker performing the procedure. In most cases, in order to perform a blood collection procedure, the healthcare worker first obtains tools and other supplies from a storage area. The healthcare worker then transports the supplies to a patient's bedside either in one trip or multiple trips. Once all needed tools and accessories are at the patient's bedside, the healthcare worker performs the blood draw and collection procedure and introduces an obtained blood sample into one or more sample containers or tubes. Tools needed for performing the blood draw and collection procedure can include, for example: syringe(s) containing flushing solutions for flushing the patient's catheter or central line; alcohol wipes or other disinfectant devices for cleaning a catheter hub, syringes, and any other devices used during the medical procedure; sample collection tubes for storing the blood sample; and/or blood transfer sets for introducing the obtained blood sample into the sample collection tubes.

Performing the blood draw and collection procedure can include performing actions, such as: disinfecting a catheter hub using an alcohol swab; attaching a syringe containing a flushing solution to the patient's catheter or central line; expelling the flushing solution from the syringe into the patient's catheter or central line; attaching the blood sample transfer set or tube holder to the patient's catheter or central line; wasting a volume of blood into a discard tube; obtaining a blood sample through the transfer set or tube holder; flushing the patient's catheter or central line after the blood sample is obtained; and/or capping the catheter or reattaching any prior tubing (i.e., an IV line) to the patient's catheter or central line.

Many healthcare workers develop their own processes for obtaining necessary tools for and performing blood draw and collection procedures. The variability in how such procedures are performed can be a source of error increasing infection risk and/or decreasing a quality of collected blood samples. In view of the inconsistent manner in which blood collection procedures can be performed, there is a need for devices, systems, and methods that encourage healthcare workers to perform blood draw and collection procedures in a more efficient manner, while closely following specific steps for collecting blood samples. It is believed that encouraging healthcare workers to follow protocols more closely can reduce errors and improve a quality of collected samples. The containers, equipment kits, and methods of the present disclosure are intended to provide such improvements for blood collection procedures.

### SUMMARY OF THE INVENTION

According to an aspect of the disclosure, a container configured to store tools used by a healthcare worker when performing a blood draw and collection procedure includes a tray and packaging. The tray includes a plurality of chambers sized to receive the tools used by the healthcare worker for the blood draw and collection procedure. The tools include at least one disposable disinfecting device for disinfecting portions of the tools and at least one tube for storing a blood sample. The packing, when in a closed or prior-to-use position, is over openings of the plurality of chambers of the tray, thereby securely containing the tools within the chambers of the tray. In an open or ready-for-use position, the packaging is separated from the openings of the plurality of chambers of the tray so that the healthcare worker can access the tools contained within the chambers while performing the blood draw and collection procedure.

According to another aspect of the disclosure, an equipment kit containing tools used by a healthcare worker when performing a blood draw and collection procedure includes: any of the previously described containers configured to store tools used by a healthcare worker when performing a blood draw and collection procedure; the at least one disposable disinfecting device contained within at least one of the plurality of chambers of the tray; and the at least one tube configured to contain the blood sample contained within another of the at least one plurality of chambers of the tray.

According to another aspect of the disclosure, a method of performing a blood draw and collection procedure includes removing at least a portion of the packaging of the container of any of the previously described equipment kits from the tray so that tools contained in the tray can be removed from the tray. The method further includes: disinfecting a hub or cap of a central line of a patient with the at least one disposable disinfecting device; attaching a blood sample transfer set or tube holder contained in the container of the kit to the disinfected hub or cap such that the transfer set or tube holder is in fluid communication with the central line; drawing a blood sample from the central line of the patient through the transfer set or tube holder into the at least one tube, thereby providing the blood sample obtained from the central line of the patient; and detaching the blood sample transfer set or tube holder from the hub or cap.

Non-limiting illustrative examples of embodiments of the present disclosure will now be described in the following numbered clauses:

Clause 1: A container configured to store tools used by a healthcare worker when performing a blood draw and collection procedure, the container comprising: a tray comprising a plurality of chambers sized to receive the tools used by the healthcare worker for the blood draw and collection procedure, the tools comprising at least one disposable disinfecting device for disinfecting portions of the tools and at least one tube for storing a blood sample; and packaging that, in a closed or prior-to-use position, is over openings of the plurality of chambers of the tray, thereby securely containing the tools within the chambers of the tray and, in an open or ready-for-use position, is separated from the openings of the plurality of chambers of the tray so that the healthcare worker can access the tools contained within the chambers while performing the blood draw and collection procedure.

Clause 2: The container of clause 1, wherein at least one of the plurality of chambers is a waste chamber configured to receive the at least one disposable disinfecting device after use.

Clause 3: The container of clause 1 or clause 2, wherein the tray further comprises a plurality of tube holders sized to receive tubes to support the tubes in an upright position relative to the tray in an order in which the tubes are to be filled with the blood sample.

Clause 4: The container of clause 3, wherein the plurality of tube holders are formed from a plate over at least a portion of one of the plurality of chambers with circular openings having a diameter substantially matching a diameter of a cross-section of the tubes.

Clause 5: The container of any of clauses 1-4, wherein the tray comprises a molded plastic tray.

Clause 6: The container of any of clauses 1-5, wherein the packaging comprises a folded cover comprising a bottom portion, at least one side portion, and a top portion configured to swing between the closed position, in which the top portion of the cover is over the openings of the plurality of chambers of the tray, and the open position, in which the top cover is spaced apart from the openings of the plurality of chambers of the tray, so that tools and/or other items can be placed in or removed from the plurality of chambers of the tray.

Clause 7: The container of clause 6, wherein the top portion of the folded cover comprises a folded flap configured to be engaged to a slot, groove, flap, or hook on the at least one side of the folded cover, thereby securing the cover in the closed position.

Clause 8: The container of clause 6 or clause 7, further comprising one or more fasteners for mounting the tray to the bottom portion of the folded cover.

Clause 9: The container of any of clauses 1-8, wherein the packaging comprises at least one removable cover sheet that is removably adhered to a top surface of the tray to secure the tools within the plurality of chambers of the tray.

Clause 10: The container of clause 9, wherein the at least one cover sheet comprises a first cover sheet over one or more of the plurality of chambers, and a second cover sheet over other chambers, and wherein the first cover sheet and the second cover sheet are separately removable from the tray.

Clause 11: The container of clause 10, further comprising a plate sized to fit within the opening of one of the plurality of chambers to prevent items from falling out of the chamber when the container is inverted.

Clause 12: The container of any of clauses 1-11, wherein the blood draw and collection procedure comprises a procedure of collecting blood from a catheter of a patient.

Clause 13: The container of clause 12, wherein the tools further comprise one or more of: at least one syringe containing a solution, such as saline or heparin, for flushing the catheter of the patient; at least one of a blood sample transfer set or tube holder configured to be engaged to the at least one tube for filling the tube with the blood sample; or the disposable disinfecting device comprises a swab or brush comprising alcohol for disinfecting the tools and/or components of a vascular tube holder connected to the patient.

Clause 14: An equipment kit containing tools used by a healthcare worker when performing a blood draw and collection procedure, the kit comprising: the container of any of clauses 1-13, the at least one disposable disinfecting device contained within at least one of the plurality of chambers of the tray; and the at least one tube configured to contain the blood sample contained within another of the at least one plurality of chambers of the tray.

Clause 15: The kit of clause 14, further comprising at least one instruction card with instructions for one or more of preparing the tools for use prior to performing the blood draw and collection procedure, flushing fluid through a catheter of a patient prior to performing the blood draw and collection, wasting a sufficient amount of blood prior to performing the blood draw and collection, filling a plurality of the tubes with a blood sample in a correct order of tubes, and/or capping the catheter of the patient after drawing blood from the patient.

Clause 16: The kit of clause 14 or clause 15, wherein the blood draw and collection procedure comprises a procedure of collecting blood from a catheter of a patient, and at least one of a blood sample transfer set or tube holder configured to be engaged to the at least one tube for filling the tube with the blood sample.

Clause 17: The kit of any of clauses 14-16, wherein the tools further comprise at least one syringe containing a solution, such as saline or heparin, for flushing a patient line.

Clause 18: The kit of any of clauses 14-17, wherein the disposable disinfecting device comprises a swab or brush comprising alcohol for disinfecting components of the tools and/or of a vascular tube holder connected to the patient.

Clause 19: A method of performing a blood draw and collection procedure, comprising: removing at least a portion of the packaging of the container of the kit of any of clauses 14-18 from the tray so that tools contained in the tray can be removed from the tray; disinfecting a hub or cap of a central line of a patient with the at least one disposable disinfecting device; attaching a blood sample transfer set or tube holder contained in the container of the kit to the disinfected hub or cap such that the transfer set or tube holder is in fluid communication with the central line; drawing a blood sample from the central line of the patient through the transfer set or tube holder into the at least one tube, thereby providing the blood sample obtained from the central line of the patient; and detaching the blood sample transfer set or tube holder from the hub or cap.

Clause 20: The method of clause 19, wherein the kit further comprises at least two syringes, each containing a solution, such as saline or heparin, for flushing the central line of the patient, the method further comprising flushing the central line of the patient with the syringes containing the flushing solution both before attaching the blood sample transfer set or tube holder to the hub or cap and after detaching the blood sample transfer set or tube holder from the hub or cap.

### BRIEF DESCRIPTION OF THE DRAWINGS

FIG. 1A is a perspective view of an equipment kit containing tools for performing a blood draw and collection procedure in a closed or prior-to-use position, according to an aspect of the present disclosure.

FIG. 1B is a perspective view of the equipment kit of FIG. 1A in an open or ready-for-use position.

FIG. 1C is a cross-sectional view of the closed or prior-to-use equipment kit of FIG. 1A taken along line 1C-1C.

FIG. 1D is another cross-sectional view of the closed or prior-to-use equipment kit of FIG. 1A taken along line 1D-1D.

FIG. 1E is a top view of the closed or prior-to-use equipment kit of FIG. 1A.

FIG. 1F is a side view of the closed or prior-to-use equipment kit of FIG. 1A.

FIG. 1G is another cross-sectional view of the closed or prior-to-use equipment kit of FIG. 1A taken along line 1G-1G.

FIG. 1H is another cross-sectional view of the closed or prior-to-use equipment kit of FIG. 1A taken along line 1H-1H.

FIG. 2A is a perspective view of another equipment kit in a closed or prior-to-use position being grasped by a user, according to an aspect of the present disclosure.

FIG. 2B is another perspective view of the equipment kit of FIG. 2A in an open or ready-for-use position upon first opening the kit, according to an aspect of the present disclosure.

FIG. 2C is another perspective view of the open kit of FIG. 2B showing a user removing tools from the open kit, according to an aspect of the present disclosure.

FIG. 3 is a perspective of another equipment kit in an open or ready-for-use position, according to an aspect of the present disclosure.

FIG. 4A is a schematic drawing of another equipment kit showing closing of the kit, according to an aspect of the present disclosure.

FIG. 4B is a cross-sectional view showing a tamper resistant closure of the equipment kit of FIG. 4A, according to an aspect of the present disclosure.

FIG. 5A is a schematic drawing of another equipment kit showing closing of the kit, according to an aspect of the present disclosure.

FIG. 5B is a cross-sectional view showing a tamper resistant closure of the equipment kit of FIG. 5B, according to an aspect of the present disclosure.

FIG. 6 is a perspective exploded view of another equipment kit, according to an aspect of the present disclosure.

FIGS. 7A and 7B show instruction cards that can be included with equipment kits including instructions to be performed by healthcare workers to perform blood draw and collection procedures, according to aspects of the present disclosure.

FIGS. 7C and 7D are photographs of equipment kits including the instruction cards of FIGS. 7A and 7B, according to aspects of the present disclosure.

FIGS. 7E and 7F show additional examples of instruction cards that can be included with equipment kits including instructions to be performed by healthcare workers to perform blood draw and collection procedures, according to aspects of the present disclosure.

FIG. 8A is a perspective view of another equipment kit in a closed or prior-to-use position, according to an aspect of the present disclosure.

FIG. 8B is another perspective view of the equipment kit of FIG. 8A in a partially open position.

FIG. 8C is another perspective view of the equipment kit of FIG. 8A in another partially open position.

FIG. 8D is another perspective view of the equipment kit of FIG. 8A in the fully open or ready-for-use position.

FIG. 8E is a top view of the equipment kit of FIG. 8A in the closed or prior-to-use position.

FIG. 8F is a front view of the equipment kit of FIG. 8A in the closed or prior-to-use position.

FIG. 8G is a side view of the equipment kit of FIG. 8A in the closed or prior-to-use position.

FIG. 9A is a perspective view of another equipment kit in a closed or prior-to-use position being grasped by a user, according to an aspect of the present disclosure.

FIG. 9B is another perspective view of the equipment kit of FIG. 9A upon first opening the kit, according to an aspect of the present disclosure.

FIG. 9C is another perspective view of the open kit of FIG. 9B showing a user removing tools from the kit, according to an aspect of the present disclosure.

FIG. 10A is a perspective view of another equipment kit in an open or ready-for-use position, according to an aspect of the present disclosure.

FIG. 10B is another perspective view of the equipment kit of FIG. 10A showing the kit in an open and in-use position, according to an aspect of the present disclosure.

FIGS. 11A and 11B show additional examples of instruction cards that can be included with equipment kits including instructions to be performed by healthcare workers to perform blood draw and collection procedures, according to aspects of the present disclosure.

FIGS. 11C and 11D show additional examples of instruction cards that can be included with equipment kits including instructions to be performed by healthcare workers to perform blood draw and collection procedures, according to aspects of the present disclosure.

FIGS. 12A and 12B are flow charts of steps for performing a blood draw and collection procedure using an equipment kit, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”. “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to packaging, containers, boxes, and equipment or toolkits including tools used for performing medical procedures, namely blood draw and collection procedures, in an efficient and accurate manner. The present inventors have recognized that healthcare workers who are responsible for acquiring different tools needed for performance of such procedures may not efficiently obtain all needed tools and accessories for performing the procedure. In such cases, healthcare workers may attempt to perform the medical procedures without all necessary tools and accessories, rather than making another trip to a storage area to obtain a required item.

The containers 10 and/or equipment kits 110 of the present disclosure are intended to provide a single easily transportable item that can be quickly obtained from a storage area and carried to a patient's bedside to perform the blood draw and collection procedure. The containers 10 and kits 110 of the present disclosure aim to reduce the risk of infection, improve quality of collected samples, and increase efficiency of blood collection procedures, such as blood collection from a catheter, compared to current practices, where healthcare workers may not always obtain all tools for the procedure in an efficient manner. In particular, the containers 10 and equipment kits 110 of the present disclosure ensure that all tools and accessories for the procedure are available to the healthcare worker before the healthcare worker begins to perform the procedure. Further, the containers 10 and kits 110 of the present disclosure can include both text and graphic instructions that reinforce proper blood collection techniques, reducing the possibility that the healthcare worker will skip steps of the procedure, perform steps of the procedure in an incorrect order, or perform steps of the procedure using the wrong tools. Accordingly, the containers 10 and equipment kits 110 of the present disclosure reduce occurrences of errors thereby improve quality and safety of blood draw and collection procedures.

Equipment Kit with Folded Cover

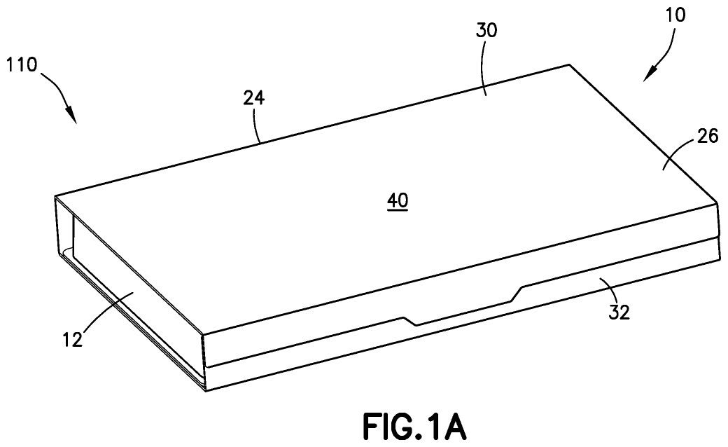

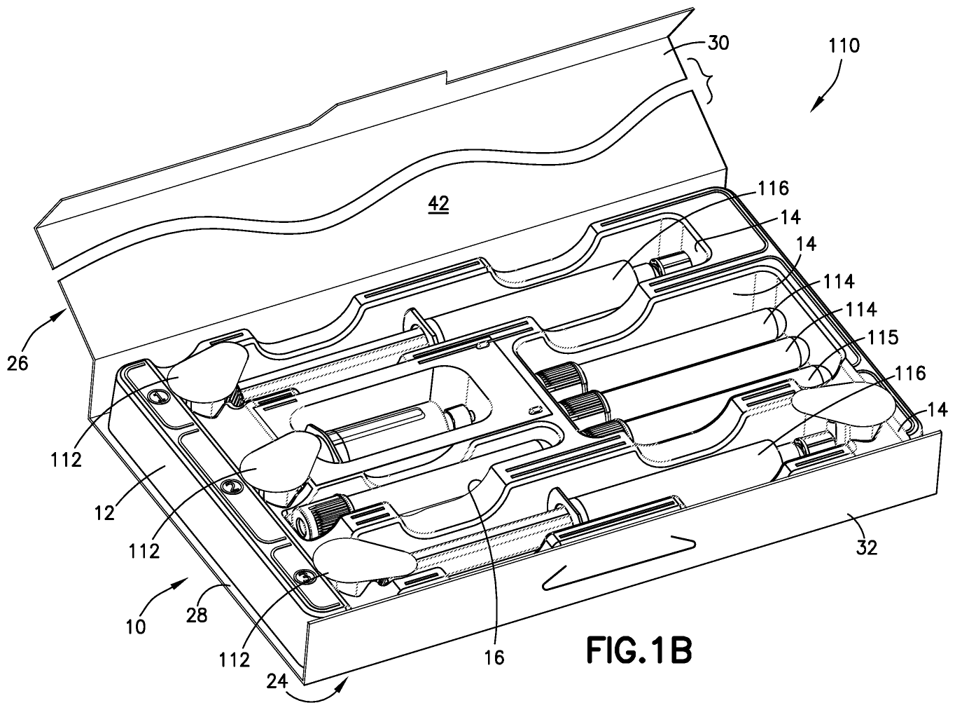

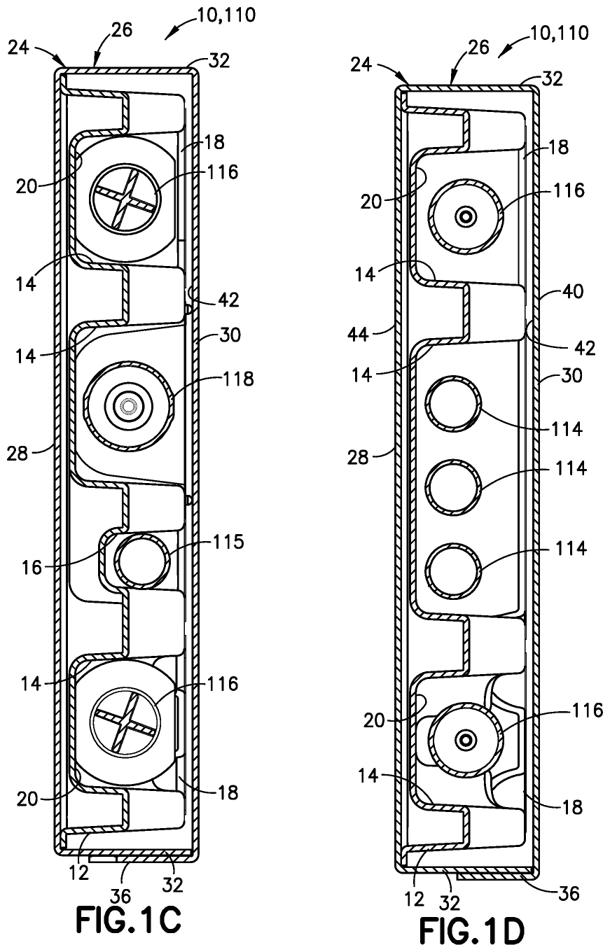

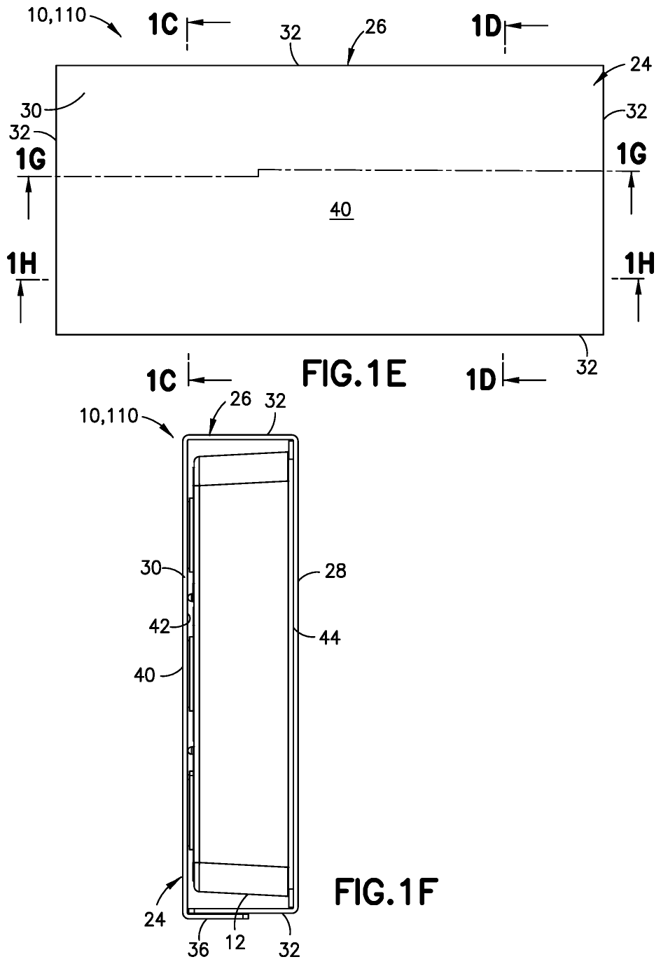

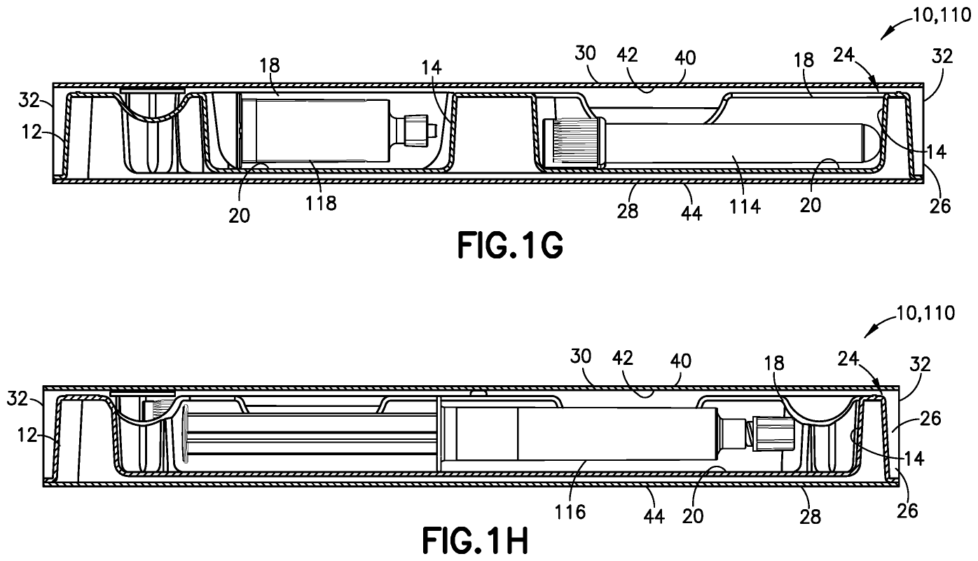

With reference to FIGS. 1A-1H, an equipment kit 110 containing tools used by a healthcare worker when performing a blood draw and collection procedure includes a container 10, box, packaging, or similar holder containing the tools for performing the blood draw and collection procedure. As used herein, a “healthcare worker” can be a medical professional trained to perform a blood collection procedure, such as a phlebotomist or nurse. The kits 110 of the present disclosure can also be used by healthcare workers without specific training for performing blood collection procedures, such as a pharmacist, physical therapist, occupational therapist, physician assistants, physicians, or another healthcare worker.

The blood draw and collection procedure can be a blood draw and collection procedure, such as blood collection from a catheter or central line, which can be performed by a healthcare worker for a patient. As described in further detail herein, tools for performing the blood draw and collection procedure can include, for example, a disposable disinfecting device 112, such as an alcohol wipe or swab, for disinfecting portions of tools in the equipment kit 110, as well as for disinfecting other medical devices or connectors including patient ports, catheter hubs, and similar devices. The disposable disinfecting device 112 can also be used for cleaning an area of the patient's skin prior to performing, for example, a venous puncture.

In some examples, the disposable disinfecting device 112 can be a cleaning device specifically designed for cleaning medical equipment. For example, the disposable disinfecting device 112 can be the Site-Scrub device manufactured by Becton, Dickinson and Company, for disinfecting female luer hubs, injection ports, and similar connectors. The Site-Scrub device is a cap-shaped device sized to fit over a catheter hub or female luer lock. The Site-Scrub device includes an interior with a foam material saturated with a disinfecting cleaner solution. The Site-Scrub device can be inserted over the catheter hub or luer lock, such that the bristles and disinfecting cleaning solution contact the hub to clean and disinfect the hub. As previously discussed, in other example, alcohol wipes and similar cleaning tissues can be used for cleaning patient ports and connectors prior to performing the blood draw and collection procedure.

The tools contained within the container 10 can further include one or multiple sample collection tubes 114 for containing a patient sample, such as a blood sample. The sample collection tubes 114 can be any suitable blood collection tube or another container, such as a vacuum-based container, with an interior volume sufficient for containing a blood sample of a desired volume, as are known in the art. In some examples, the sample tubes 114 can be a vacuum-based container, such as the BD Vacutainer® sample collection tube manufactured by Becton, Dickinson and Company, available in plastic or glass. In other examples, other types of tubes or containers, which may or may not be vacuum sealed, can be used within the scope of the present disclosure. The sample collection tubes 114 can include and/or be coated with various blood preservation agents, reagents, and other chemicals to preserve the sample and/or so that the sample can be used for specific tests, as are known in the art. The container 10 can also include a waste or discard tube 115 that receives a waste amount of the patient's blood before the blood sample is collected. The waste or discard tube 115 can be similar in shape and structure to the other sample collection tubes 114. For example, the waste or discard tube 115 can be a BD Vacutainer® tube. In some examples, the waste or discard tube 115 can be smaller (i.e., can contain a smaller volume) than the sample collection tubes 114. Further, the waste or discard tube 115 can include coloration or markings indicating that it is to be used to collect a waste amount of blood and not a blood sample to be used for a blood test.

The kit 110 can also include syringes 116, such as pre-filled syringes containing a flushing solution, such as saline or heparin. The syringes 116 containing the flushing solution are configured to be attached to a hub of the patient's catheter or central line, thereby establishing fluid communication between a barrel of the syringe 116 and the patient's catheter or central line. The flushing solution can be expelled from the syringe 116, through the catheter or central line, to the vasculature of the patient. The flushing solution is provided to ensure that the patient's catheter or central line is clean, thereby avoiding contamination of the collected blood sample. The patient's catheter or central line can be flushed both before and after the blood sample is obtained. Many different syringes 116 commercially available from different manufacturers can be used for providing the flushing solution through the catheter or central line of the patient. In some examples, the syringe 116 is the BD PosiFlush™ pre-filled saline syringe by Becton, Dickinson and Company. In other examples, the syringe 116 can be filled with an anticoagulant, such as heparin.

Another tool that can be contained in the kit 110 is a blood sample transfer set or tube holder 118 configured to be engaged to the waste or discard tube 115 and/or sample collection tubes 114 for filling or partially filling the sample collection tubes 114 with the blood sample. For example, the blood sample transfer set or tube holder 118 can be the Vacutainer® Luer-Lok™ Access Device manufactured by Becton, Dickinson and Company. In some examples, the transfer set or tube holder 118 can be connected directly to a hub of the patient's catheter or central line, so that sample tubes 114, 115 can be filled directly from the patient's catheter or central line. In other examples, a blood sample can be drawn into a syringe 116. The syringe 116 can then be connected to one of the sample collection tubes 114 by connecting a needle to a distal end of the syringe using the transfer set or tube holder 118, such as using the Vacutainer® Blood Transfer Device by Becton, Dickinson and Company. Once the syringe 116 is attached to the transfer set or tube holder 118, the blood sample can be expelled from the syringe 116 into the sample collection tubes 114 through the transfer set or tube holder 118. In some examples, the kit 110 can include multiple sample collection tubes 114, such as three, four, five, or more sample collection tubes 114. For some procedures, the tubes 114 need to be filled by the blood sample in a specific predetermined order. As described in further detail herein, the container 10 or kit 110 can include instructions, graphical icons, and tube holding structures that encourage the user to fill the sample collection tubes 114 according to the predetermined order.

The equipment kit 110 also includes the container 10 for housing the tools 112, 114, 116, 118 needed for the blood collection procedure. The container 10 is an enclosure including, for example, dividers, separators, compartments, retaining structures, and similar organizing structures for holding the tools and other accessories used for the blood collection procedure in an intuitive and convenient manner. In some examples, the container 10 is intended to be disposable, meaning that it is formed from lightweight, inexpensive materials, and can be disposed of after a single use. In other examples, the container 10 can be a reusable container that can be, for example, cleaned, sterilized, and refilled with tools and other accessories following each procedure, so that the container 10 can be reused. Desirably, the container 10 is arranged to contain the tools and other accessories used for the blood draw and collection procedure in an intuitive and clear arrangement that encourages the healthcare worker to perform the blood draw and collection procedure in accordance with accepted protocols. For example, tools can be arranged in order of use, with the tool that is used first during the blood draw and collection procedure stored at the top or front of the container 10 and the tool that is used last stored near the bottom or rear portion of the of the container. In other examples, the tool that is used first during the blood draw and collection procedure can be stored on, for example, a left side of the container 10 and the tool that is used last during the blood draw and collection procedure can be stored on the right side of the container 10. Alternatively, the tool that is used first can be stored near the top or right side of the container 10 and the tool that is used last can be stored near the bottom or left side of the container 10. Also, as previously discussed, the container 10 can include text instructions, graphical icons, color-coded portions, and other printed materials to encourage adherence to accepted protocols, such as to local hospital protocols or other known or widely-practiced protocols.

In some examples, the container 10 can include a tray 12 including chambers 14 sized to receive the tools used by the healthcare worker for the blood draw and collection procedure. The chambers 14 are generally sized to correspond to sizes of different tools and accessories used for blood draw and collection procedures. For example, the tray 12 can include elongated chambers 14 sized to receive cylindrical or tubular items, such as the syringes 116 or sample collection tubes 114. The tray 12 can also include smaller chambers 14 for receiving accessories, such as the disinfectant wipes or disposable disinfecting devices 112. In some examples, one or more of the chambers 14 are waste chambers 16 configured to receive disposable accessories, such as the disposable disinfecting devices 112, after use. The chambers 14, 16 are generally open structures having an open top 18 and a closed bottom 20, as shown in FIGS. 1G and 1H. The tray 12 can be formed from a suitable material, that is lightweight, but sufficiently rigid to maintain its shape when, for example, equipment kits 110 are stacked on top of one another in a medical facility storage area. For example, the tray 12 can be made from various hard or flexible plastics, as are known in the art. In some examples, the tray 12 can be a plastic structure formed by a suitable molding process, such as by injection molding or thermoforming.

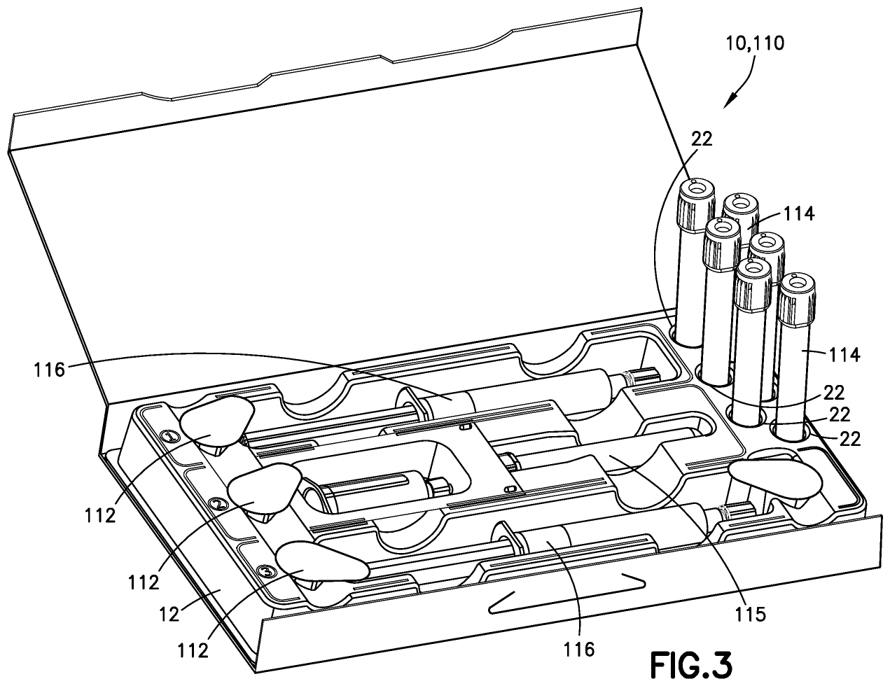

In some examples, as shown in FIG. 3, the tray 12 can further comprise tube stands or tube holders 22 sized to receive the sample collection tubes 114 and to support the sample collection tubes 114 in an upright position relative to the tray 12. For example, sample collection tubes 114 can be inserted into the tube holders 22, prior to obtaining the blood sample, in the order in which the tubes 114 should be filled with blood. By lining up the tubes 114 in a conspicuous, upright position, the healthcare worker is more likely to obtain all required blood samples in the correct order than if tubes 114 were left in chambers 14, 16 or in other less conspicuous portions of the tray 12.

In some examples, the tube holders 22 can include circular openings having a diameter that is substantially equivalent (e.g., within 5%, 10%, or 20%) of the outer diameter of the sample collection tubes 114, so that the sample collection tubes 114 can be inserted through the openings and held in the upright position, as shown in FIG. 3. Further, there can be graphical icons, text instructions, and similar content printed in proximity to the tube stands or tube holders 22 to assist and provide guidance for the healthcare worker in filling the sample collection tubes 114 with the blood sample in a correct order. For example, sample collection tubes 114 can have caps or labels of specific colors indicating the type of reagent in the sample collection tube 114. Stickers, labels, or other indicators can be positioned on portions of the tray 12 proximate to each tube stand or tube holder 22. The stickers, labels, or other indicators can comprise and/or can be printed with a color of one of the sample collection tubes 114, indicating which tube 114 should be inserted into each tube stand or holder 22, thereby encouraging the healthcare worker to place the correct sample collection tube 114 in the correct holder 22.

The container 10 also includes packaging 24 surrounding, enclosing, and/or covering the tray 12. The container 10 can be initially provided in a closed or prior-to-use configuration (shown in FIGS. 1A and 1E-1H) where the packaging 24 is over the chambers 14, 16 of the tray 12 to securely contain the tools within the chambers 14, 16 of the tray 12. As used herein, securely containing or holding the tools and accessories within the chambers 14, 16 means that the packaging 24 is held to the tray 12 tightly enough that the container 10 can be jostled, shaken, inverted, rotated, or otherwise moved without tools falling out of the container 10. The tools can be removed from the chambers 14, 16 by, for example, pulling the packaging 24 away from the chambers 14, 16, ripping or otherwise deforming the packaging 24, or similar actions. Once the packaging 24 is fully or partially removed from the tray 12 or otherwise separated from the chambers 14, 16, the container 10 is in an open or ready-for-use position (shown in FIG. 1B), where the healthcare worker can access the tools contained within the chambers 14, 16, and can begin performing the blood draw and collection procedure.

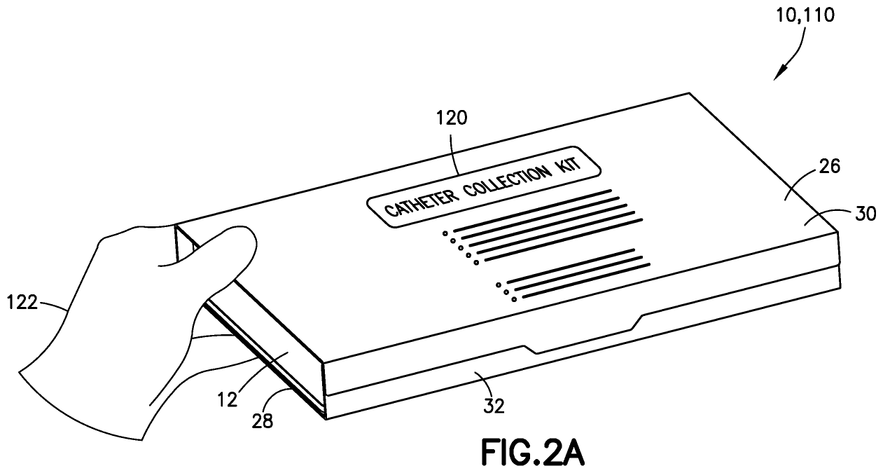

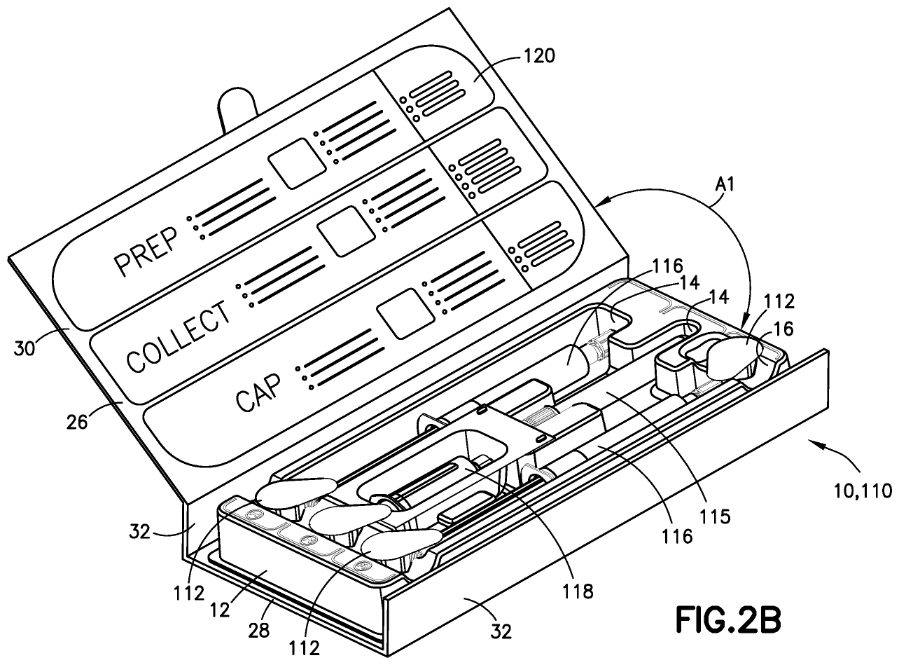

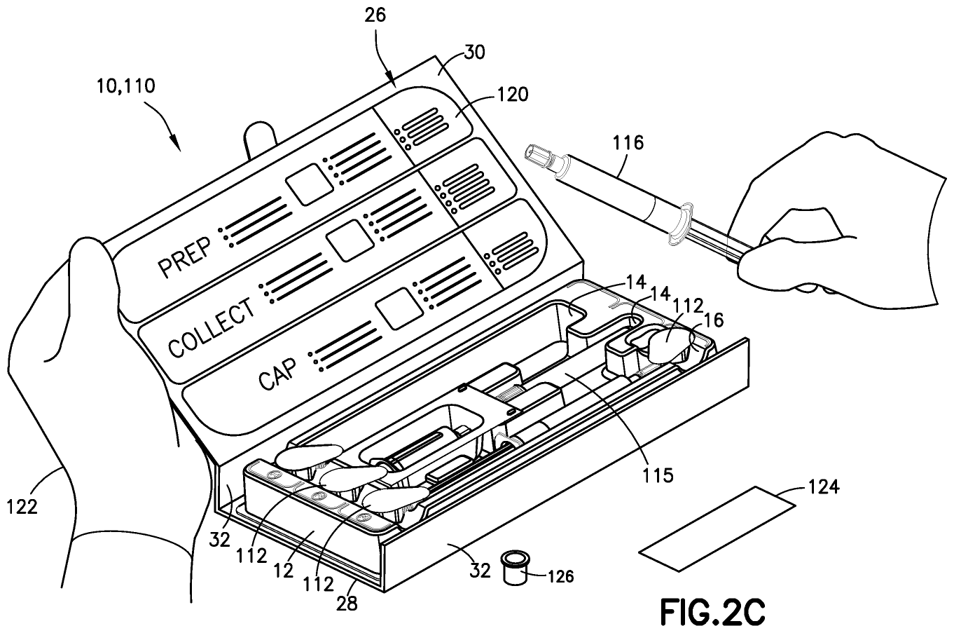

In some examples, the packaging 24 includes a folded cover 26, forming a book or housing, at least partially enclosing the tray 12. The folded cover 26 can be formed, for example, from cardboard that has been scored at certain fold points so that the cover 26 can be folded over the tray 12 to cover the openings 18 of the chambers 14, 16. The folded cover 26 can include, for example, a bottom portion 28 configured to be positioned under a bottom of the tray 12, a top portion 30 configured to be positioned over the openings 18 of the chambers 14, 16 of the tray 12 when in the closed position, and one or more side portions 32 extending between the bottom portion 28 and the top portion 30 of the folded cover 26. The top portion 30 is configured to swing, as shown by arrow A1 (shown in FIG. 2B), between the closed position (shown in FIGS. 1A and 1C-1H), in which the top portion 30 of the cover 26 is over the openings 18 of the chambers 14, 16 of the tray 12, and the open position (shown in FIGS. 1B and 2B) in which the top portion 30 of the folded cover 26 is spaced apart from the openings 18 of the chambers 14, 16 of the tray 12, so that tools and/or other items can be accessed, placed in, or removed from the chambers 14, 16 of the tray 12.

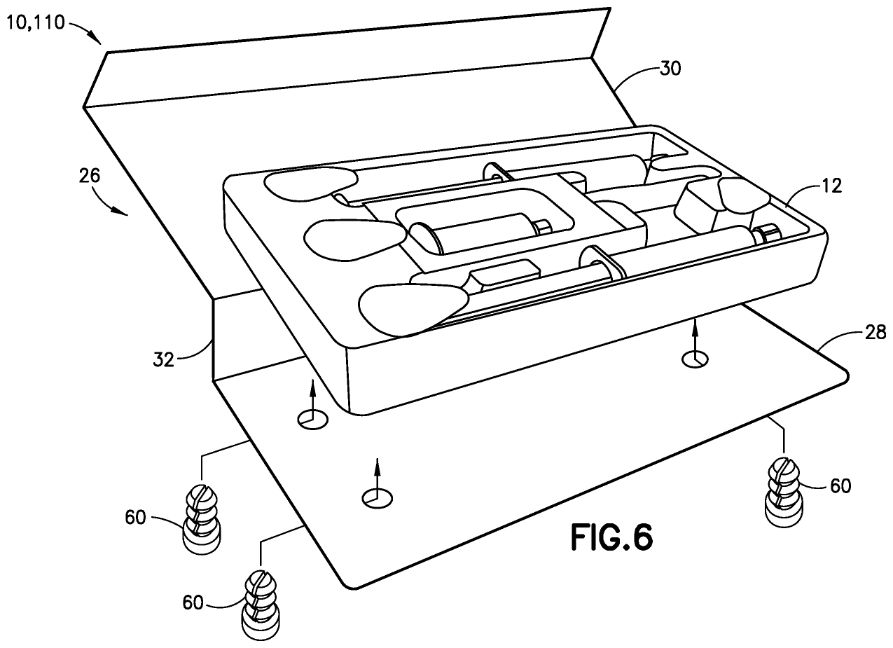

The tray 12 can be connected to the folded cover 26 by any convenient adhesive, fastener, or connector, as are known in the art. For example, common adhesives or double sided tape can be used for attaching the tray 12 to the bottom portion 28 of the folded cover 26. In other examples, mechanical fasteners, such as screws, bolts, staples, rivets, and similar connectors can be used for attaching the tray 12 to the folded cover 26. For example, as shown in FIG. 6, plastic screws 60 extending through the bottom portion 28 of the folded cover 26 can be used for connecting the tray 12 to the folded cover 26.

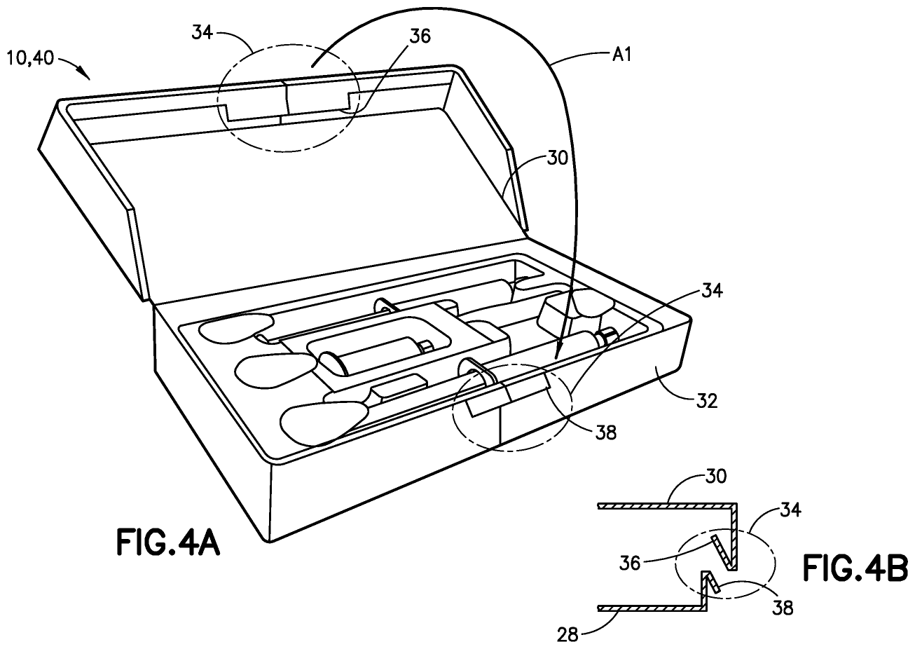

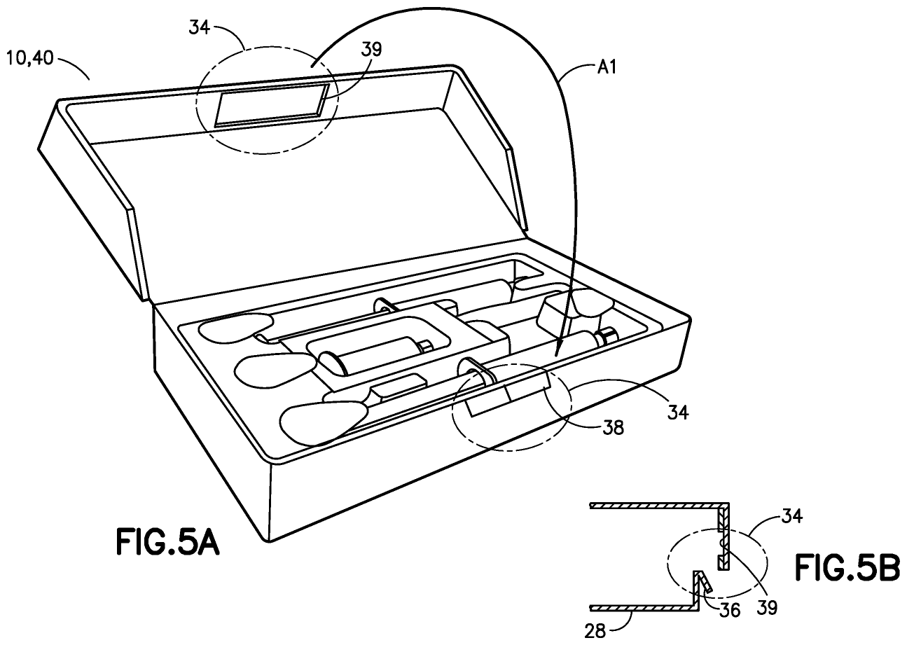

With reference to FIGS. 4A-5B, the folded cover 26 can include a tamper resisting or tamper evidencing mechanism 34, such as a slot, flap, hook, folded area, or other structure that secures the folded cover 26 in the closed position and shows when the container 10 has been previously opened or tampered with. For example, as shown in FIGS. 4A and 4B, the top portion 30 of the folded cover 26 can include a folded flap 36 configured to be engaged to a slot, groove, flap, or hook on the side 32 of the folded cover 26, thereby securing the folded cover 26 in the closed position. Specifically, as shown in FIGS. 4A and 4B, there is an opposing flap 38 extending from the side portion 32 of the folded cover 26. The folded flap 36 on the top portion 30 and the opposing flap 38 on the side portion 32 are configured to connect together, thereby securing the folded cover 26 in the closed position. In another example, as shown in FIGS. 5A and 5B, a flap 38 extending from the side portion 32 of the folded cover 26 is configured to be received within a cut-out or groove 39 on an inner surface of the side portion 32 or top portion 30 of the folded cover 26 to secure the folded cover 26 in the closed position.

In some examples, the kit 110 further includes one or more instruction cards 120 positioned on and/or mounted to different areas of the folded cover 26, such as an outer surface 40 of the top portion 30 of the folded cover 26 (as shown in FIG. 2A), an inner surface 42 of the top portion 30 (as shown in FIGS. 2B and 2C), an outer surface 44 of the bottom portion 28, or outer surface 46 of side portions 32 of the folded cover 26. In other examples, the instruction cards 120 can be positioned, for example, in chambers 14, 16 of the tray 12. For example, the instruction cards 120 can be positioned in the chambers 14, 16 below the tools, so that the healthcare worker sees the instruction cards 120 as he or she is removing a tool from one of the chambers 14, 16 of the tray 12. The instruction card 120 can include text (i.e., written instructions for performing certain actions), as well as graphical instructions or icons that guide the healthcare worker through proper performance of the blood draw and collection procedure. For example, the instruction card(s) 120 may provide instructions for one or more of the following actions: preparing the tools for use prior to performing the blood draw and collection procedure, cleaning catheter ports, flushing fluid through a catheter or central line of the patient prior to performing the blood draw and collection procedure, attaching the blood transfer set or tube holder to a hub of the patient's central line, discarding a sufficient volume of blood through the transfer set or tube holder into a waste or discard tube prior to performing the blood draw and collection procedure, filling a plurality of the tubes with a blood sample in a correct order of tubes, and/or capping the catheter or central line of the patient after drawing blood from the patient.

FIGS. 2A-2C show steps performed by the healthcare worker when using the equipment kit 110 to perform the blood draw and collection procedure. As shown in FIG. 2A, the healthcare worker first obtains the closed equipment kit 110. The healthcare worker grasps the cover 26 with his or her hand 122 and lifts the top portion 30 of the folded cover 26 away from the tray 12 in the direction of arrow A1 (shown in FIGS. 1B and 2B), so that the kit 110 is in the open or ready-for-use position, as shown in FIG. 2B. When the equipment kit 110 is in the open position, the healthcare worker can access the syringes 116, waste or discard tube 115, sample collection tubes 114, disposable disinfecting devices 112, and any other tools stored in the container 10 of the kit 110. There is an instruction card 120 on the inner surface 42 of the top portion 30 of the folded cover 26, so that the healthcare worker is reminded of instructions for performing the blood draw and collection procedure. As shown in FIG. 2C, the healthcare worker has removed a syringe 116 from the tray 12. Also, the healthcare worker has discarded a protective packaging or sleeve 124 around the syringe 116 and removed a cap 126 from a nozzle of the syringe 116. Once the protective packaging or sleeve 124 and cap 126 are removed, the syringe 116 can be used to flush the patient's catheter or central line, as previously described.

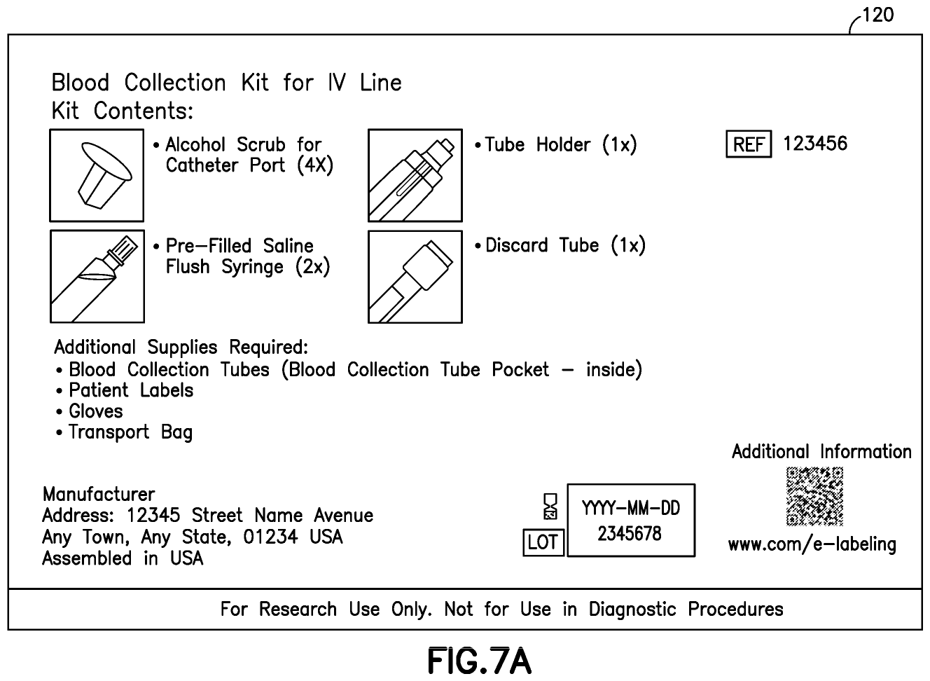

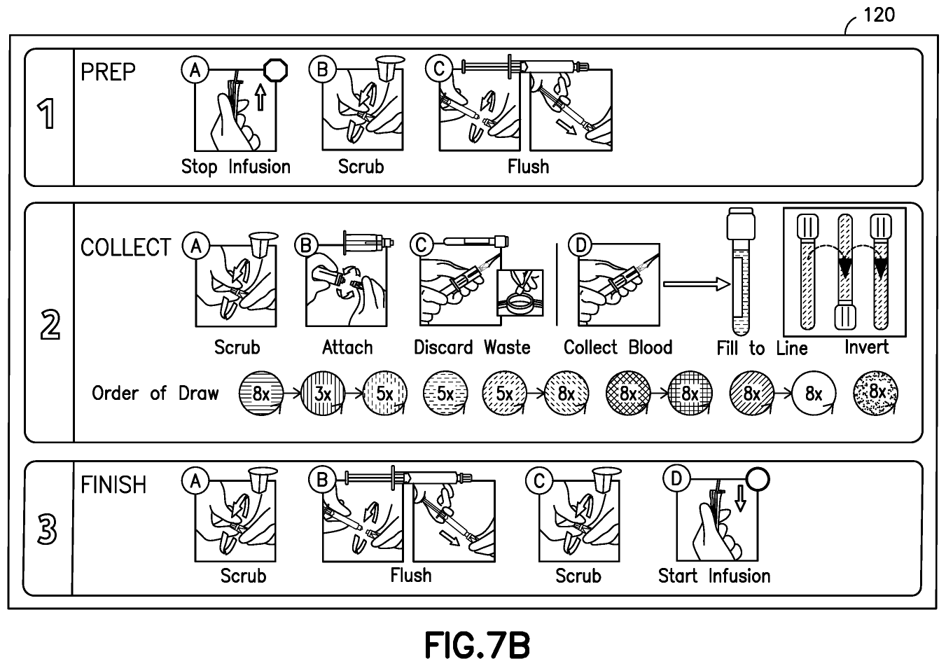

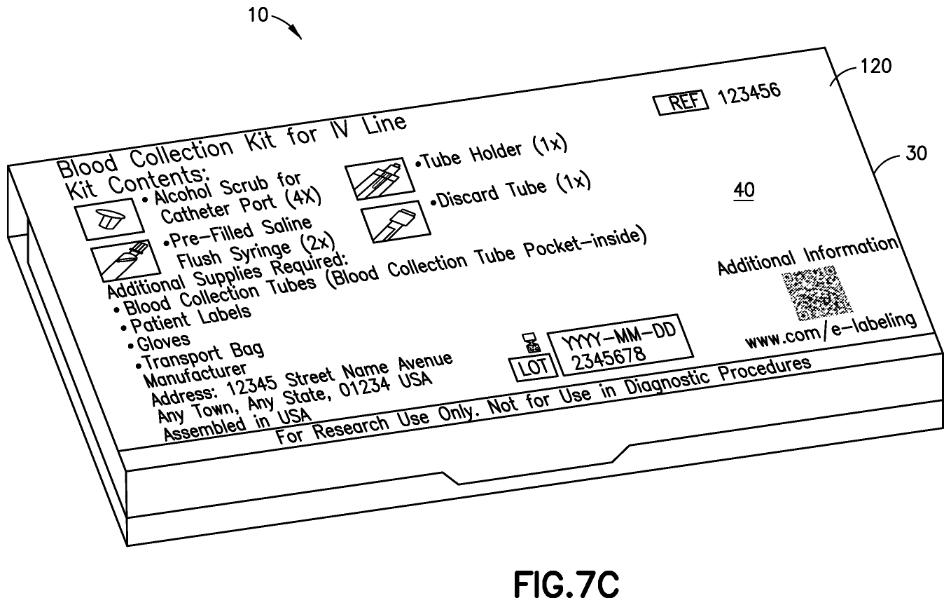

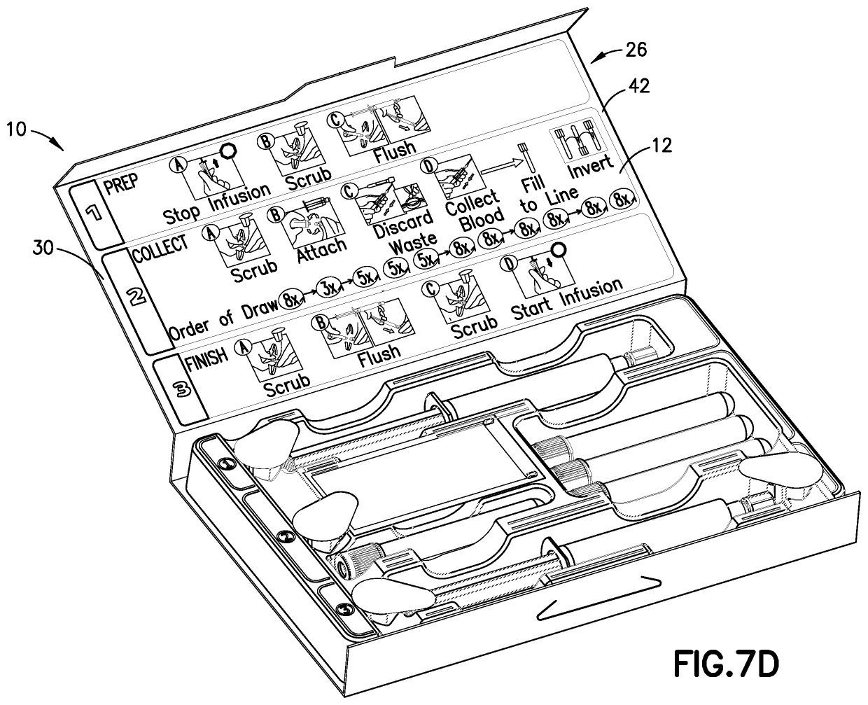

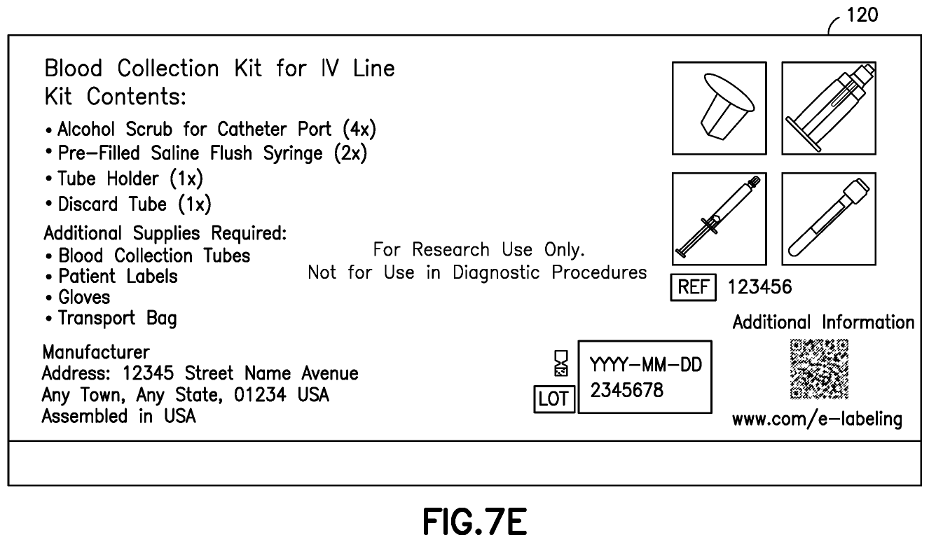

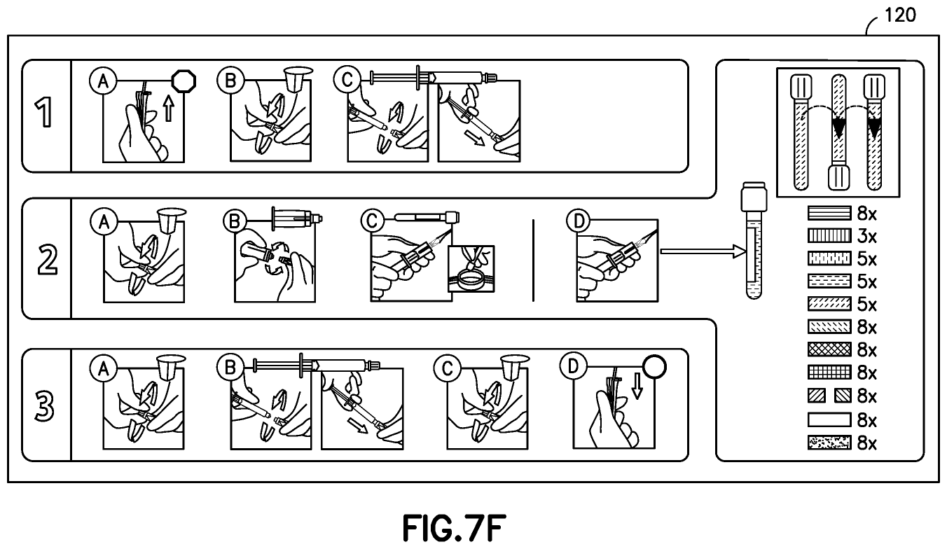

FIGS. 7A-7F show additional examples of instruction cards 120 that can be used with the containers 10 of the present disclosure. For example, FIG. 7A shows an instruction card 120 that can be adhered, for example, to an outer surface 40 of the top portion 30 of the folded cover 26. The instruction card 120 in FIG. 7A includes information that may be important for a healthcare worker to consider before beginning a blood draw and collection procedure. For example, the instruction card 120 includes a list of kit contents (e.g., four alcohol scrubs for catheter ports, one blood sample transfer set or tube holder, two pre-filled syringes, and one waste or discard tube). The instruction card 120 also includes a list of additional supplies that may be needed for the blood draw and collection procedure (e.g., blood collection tubes (if not already present in the container 10), patient labels, medical gloves, transport bags, etc.). The instruction card 120 can also include, for example, a QR code that can be used by the healthcare worker to obtain additional information about the blood draw and collection procedure to be performed.

FIG. 7B shows another example, of an instruction card 120 that can be adhered, for example, to an inner surface 42 of the top portion 30 of the cover 26 including information about specific tasks to be performed by the healthcare worker during the blood draw and collection procedure. Specifically, as shown in FIG. 7B, the instruction card 120 includes drawings or icons showing steps to be performed by the healthcare worker: during preparation for the blood draw and collection procedure (e.g., stopping an infusion, disinfecting the catheter hub, flushing the central line); while collecting the blood sample (e.g., disinfecting the catheter hub, attaching the transfer set or tube holder to the hub, wasting an amount of blood into a discard tube, collecting an appropriate amount of blood sample in each sample collection tube); and to finish the procedure (e.g., disinfecting the catheter hub, flushing the central line again, capping the hub, and restarting the infusion). FIGS. 7C and 7D are photographs showing the instruction cards 120 of FIGS. 7A and 7B mounted to the container 10 at appropriate locations.

FIGS. 7E and 7F show additional examples of instruction cards 120 that can be adhered to and/or provided with the container 10. The instruction cards 120 of FIGS. 7E and 7F include similar information to the instruction cards 120 shown in FIGS. 7A-7D, but rely primarily on graphics and drawings rather than text instructions. As in previous examples, the instruction card 120 in FIG. 7E includes information about the contents of the kit or container 10 and additional supplies required for performing the blood draw and collection procedure. The instruction card 120 of FIG. 7F includes drawings and graphic icons showing steps for preparing to perform the blood collection procedure, collecting the blood sample, and completing the procedure. The instruction card 120 of FIG. 7F also includes graphics showing instructions and reminders for how the tubes should be filled. For example, the instruction card 120 can include a reminder to invert the sample collection tubes after they are filled. The card 120 can also include a reminder to fill the tubes completely with the blood sample. The card 120 can also include a reminder about an order in which the sample collection tubes should be filled. For examples, as shown in FIG. 7F, the card 120 includes a column of rectangles filled with different colors, cross-hatch patterns, or gradients corresponding to different tubes showing the order in which the sample collection tubes should be filled. The card 120 can also include a reminder about how many times each tube should be inverted after the blood sample is collected.

Equipment Kit with Removable Cover Sheet

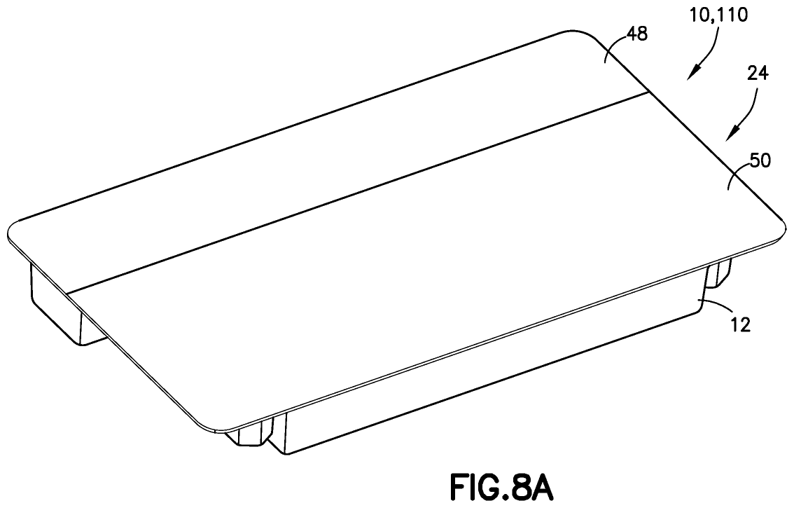

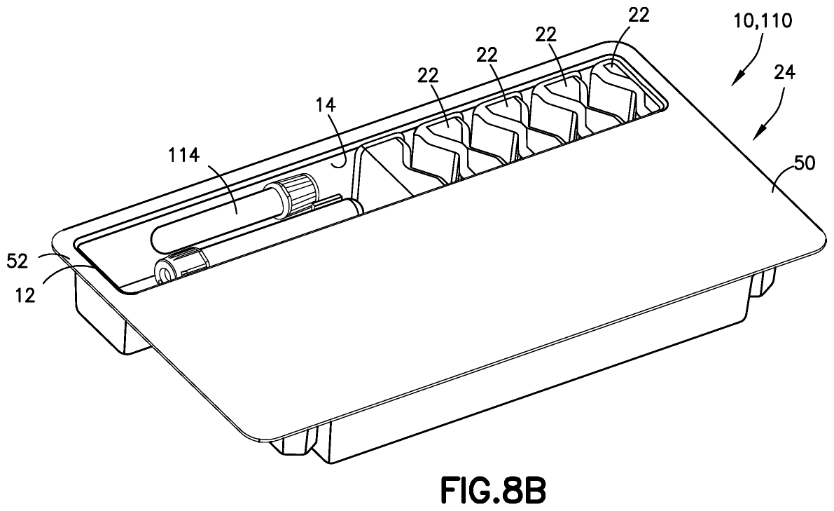

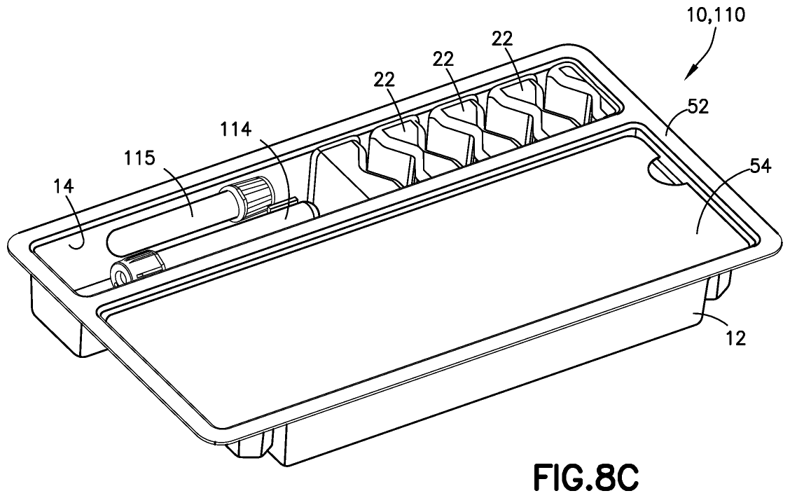

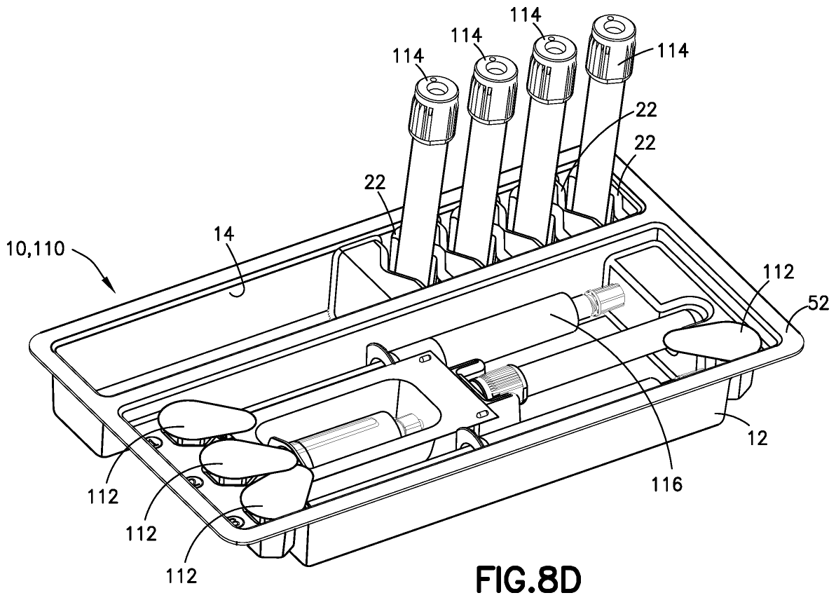

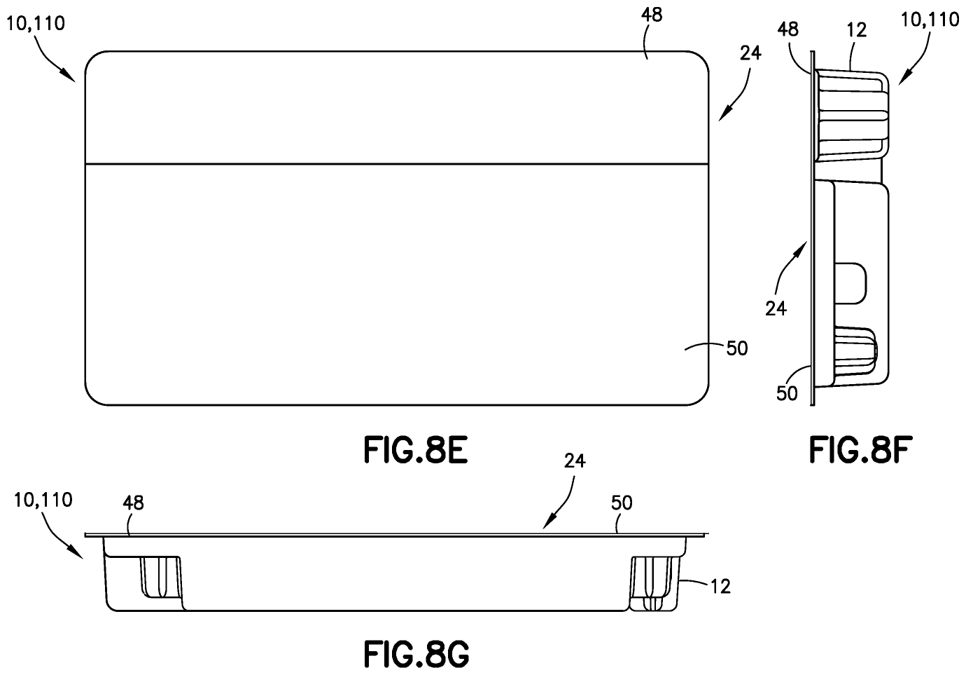

With reference to FIGS. 8A-8G, according to another example of the disclosure, the container 10 of the equipment kit 110 includes the tray 12 and packaging 24 in the form of a removable sheet 48, 50 adhered to a top surface 52 of the tray 12 with, for example, conventional adhesives used for stickers, tags, labels, and similar items, as are known in the art. The tray 12 can be similar or identical to the previously described trays 12, and can include, for example, the chambers 14 sized to contain tools and accessories used for the blood draw and collection procedure. The tray 12 can also include the one or more waste chambers 16, as previously described.

As shown in FIG. 8A, the packaging 24 can include a first removable cover sheet 48 over one or more of the chambers 14, 16 of the tray 12, and a second removable cover sheet 50 over other chambers 14, 16 of the tray 12. The first cover sheet 48 and the second cover sheet 50 are not attached to each other and are separately removable from the tray 12. Accordingly, as shown in FIG. 8B, the healthcare worker can remove the first cover sheet 48 to access tools contained within the one or more chambers 14, 16 covered by the first cover sheet 48. Once the healthcare worker has completed all tasks related to the tools contained in the chambers 14, 16 covered by the first cover sheet 48, the healthcare worker can remove the second cover sheet 50 so that, as shown in FIG. 8C, all of the chambers 14, 16 of the tray 12 are visible and accessible. As shown in FIG. 8C, one of the chambers 14 is covered by a divider plate 54 extending over a number of the tools and accessories contained within the tray 12. The divider plate 54 can be a substantially rigid plate extending over the chamber 14 that provides additional protection for tools contained therein. In particular, the divider plate 54 can be configured to prevent tools and other items from moving and/or falling out of the tray 12 at inappropriate times. As shown in FIG. 8D, the divider plate 54 has been removed, and the sample collection tubes 114, which receive the blood samples, are arranged in an upright position in the tube supports or tube holders 22 of the tray 12.

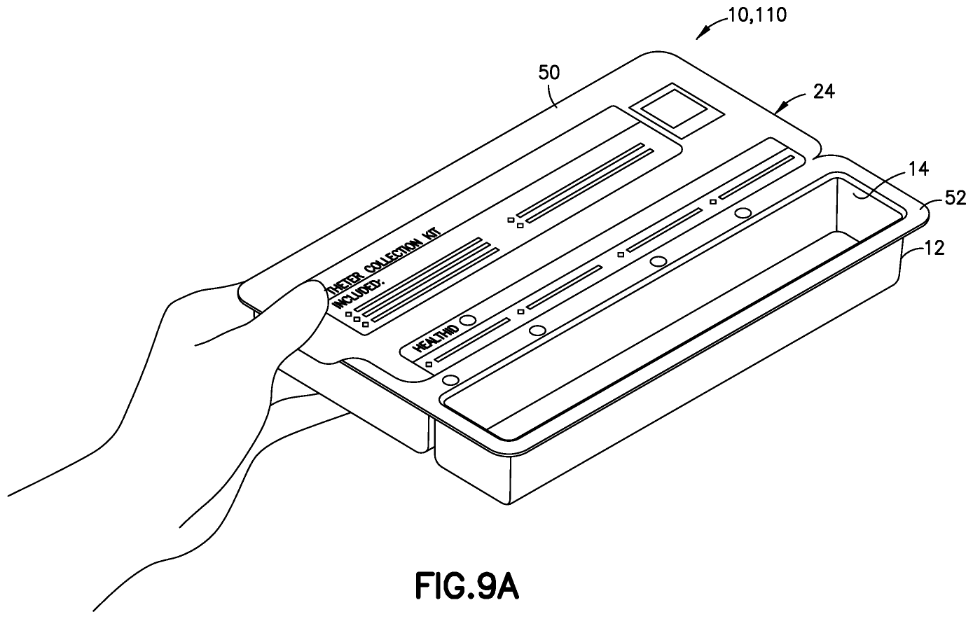

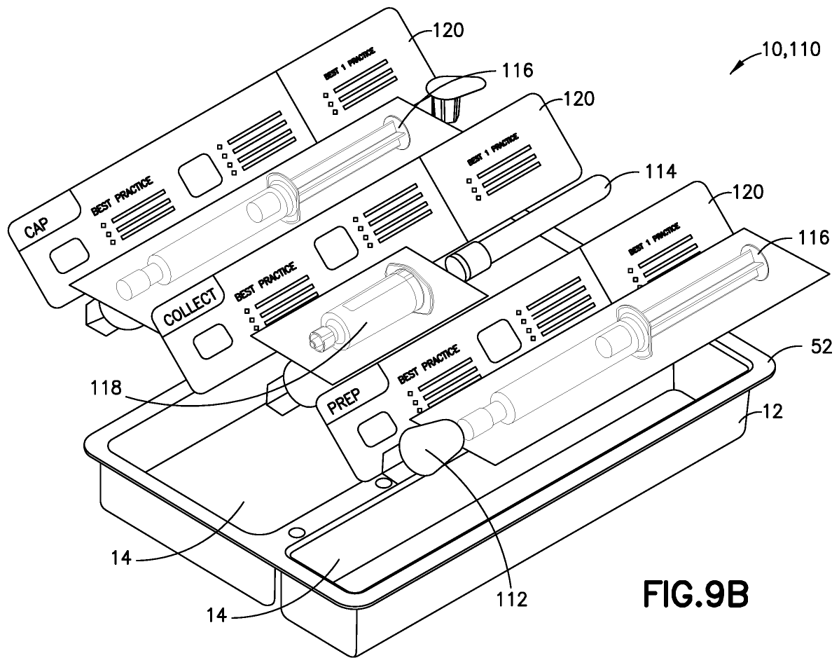

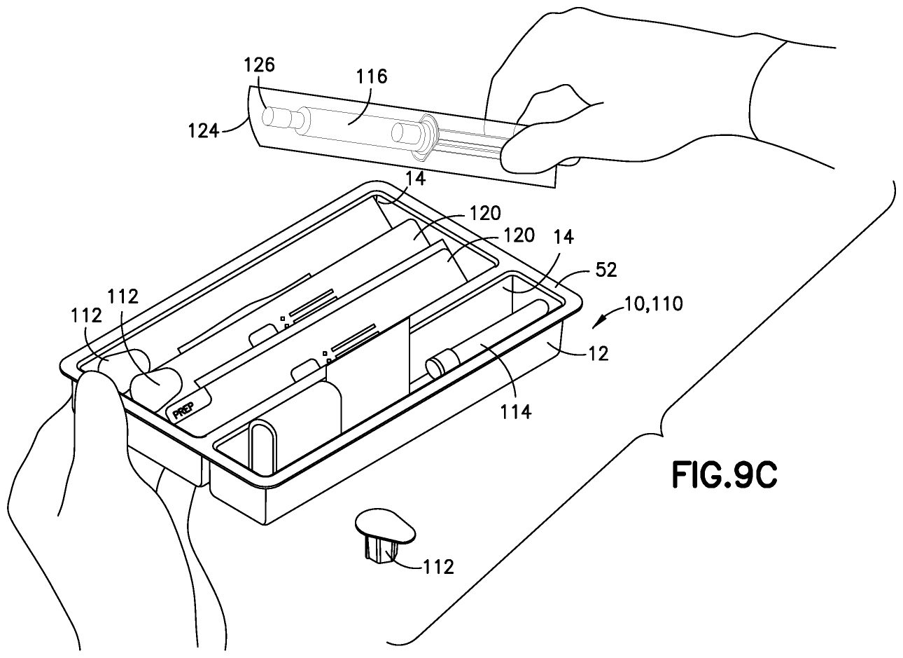

FIGS. 9A-9C are drawings showing steps performed by the healthcare worker using an exemplary equipment kit 110 including the tray 12 and the removable cover sheets 48, 50 to perform a blood draw and collection procedure. As shown in FIG. 9A, the healthcare worker has removed the first removable cover sheet 48 from the tray 12 so that the healthcare worker can access tools and accessories contained within chambers 14, 16 covered by the first cover sheet 48. FIG. 9B shows a number of different tools that are contained in the chambers 14 of the tray 12 including a syringe 116, transfer set or tube holder 118, sample collection tubes 114, and disposable disinfecting devices 112. The kit 110 also includes the instruction cards 120 with instructions for using the tools to perform blood draw and collection procedures. As shown in FIG. 9B, an instruction cards 120 can be positioned in each chamber 14 proximate to one of the tools used for the blood draw and collection procedure with instructions for use for the particular tool. FIG. 9C shows the healthcare worker removing a tool, specifically a pre-filled syringe 116 containing flushing solution, from the tray 12. The syringe 116 is enclosed in a plastic protective packaging or sleeve 124, which can be removed prior to attaching the syringe 116 to a hub of a patient's catheter or central line. Once the syringe 116 is connected to the patient's catheter or central line, the healthcare worker can expel the flushing solution from the syringe 116 to flush the patient's catheter or central line.

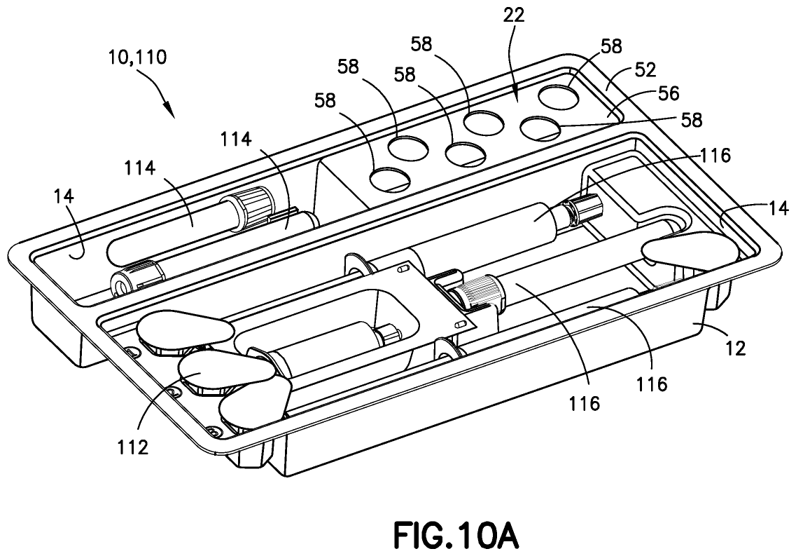

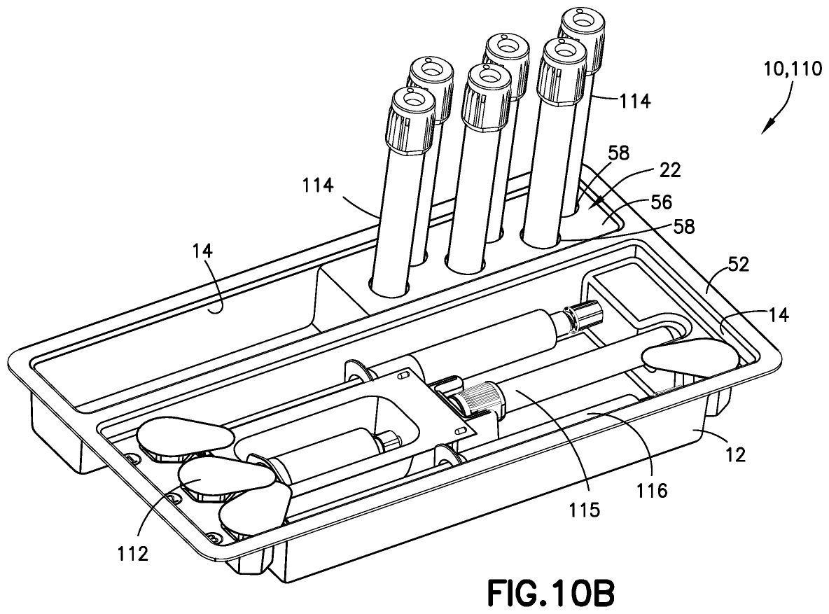

With reference to FIGS. 10A and 10B, another example of a tray 12 of the equipment kit 110 is illustrated. The tray 12 in FIGS. 10A and 10B can be used with either the kits 110 including the folded cover 26 or the kits 110 with the removable cover sheets 48, 50 adhered to the top surface 52 of the tray 12, as previously described. In either case, the tray 12 of FIGS. 10A and 10B includes a tube support or tube holder 22 for supporting the sample collection tubes 114 in an upright position. Unlike in previous examples, in which the tube supports or tube holders 22 were molded structures formed in the molded tray 12, in FIGS. 10A and 10B, the tube supports or tube holders 22 include a plate 56 over at least a portion of one of the chambers 14, 16 with circular openings 58 having a diameter substantially matching a diameter of a cross-section of the sample collection tubes 114. As shown in FIG. 10B, the sample collection tubes 114 are inserted through the openings 58 and held in the upright position by the plate 56.

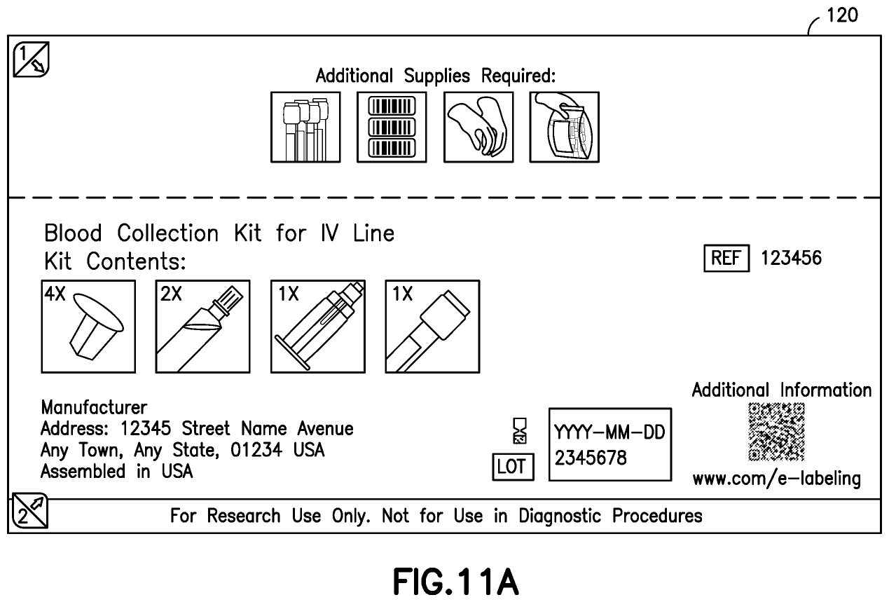

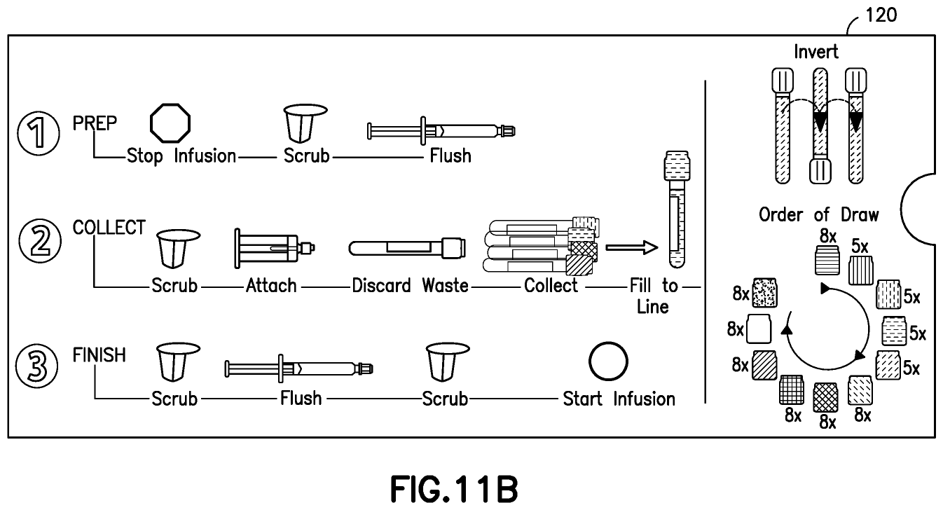

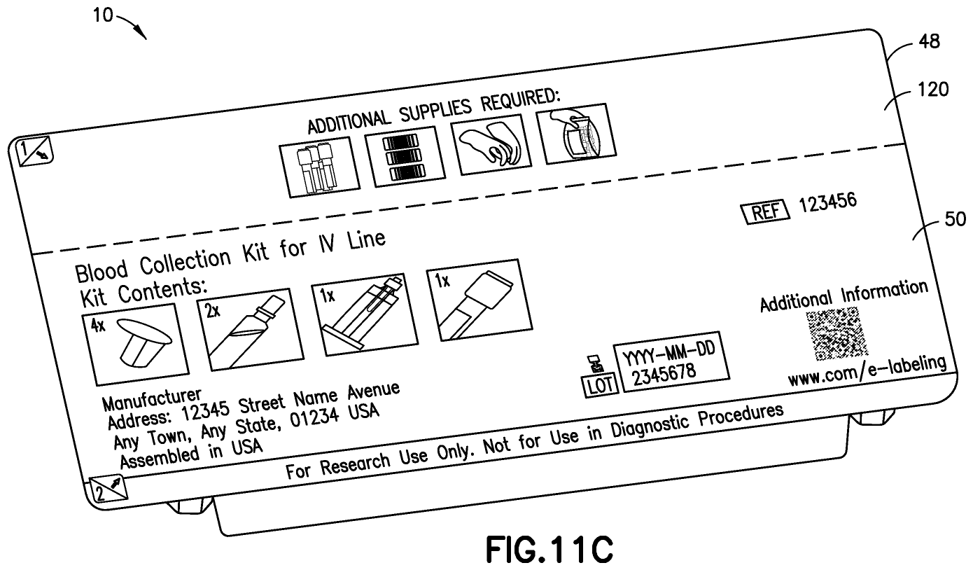

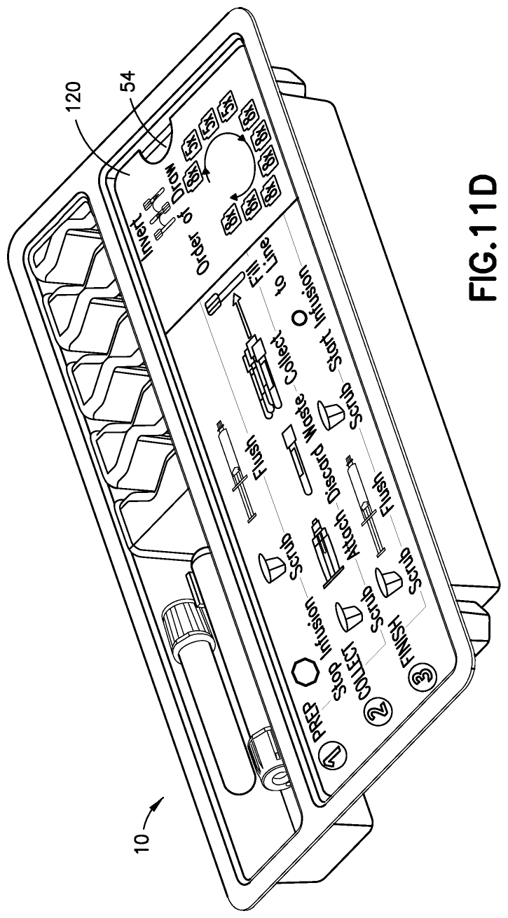

FIGS. 11A-11D show additional examples of instruction cards 120 that can be used with the containers 10 of FIGS. 8A-10B. For example, FIG. 11A shows an instruction card 120 that can be provided on and/or applied to the packaging 24 of the container 10. The instruction card 120 of FIG. 11A can be printed on the first and/or second cover sheets 48, 50 of the container 10. The instruction card 120 in FIG. 11A can include information relevant for the healthcare worker as the healthcare worker is preparing to perform the blood collection procedure. For example, as in previous examples, the instruction card 120 can include a list of items contained in the kit, information about additional supplies needed to perform the blood draw and collection procedure, and a QR code allowing the healthcare worker to access additional information about the blood draw and collection procedure.

FIG. 11B shows an instruction card 120 that can be provided inside the container 10 of FIGS. 8A-10B below the cover sheets 48, 50. For example, the instruction card 120 of FIG. 11B can be attached to and/or printed on the divider 54 shown, for example, in FIG. 8C. As in previous examples, the instruction card 120 of FIG. 11B includes text instructions and graphics showing steps for preparing to perform the blood collection procedure, collecting the blood samples, and completing the blood collection process. The instruction card 120 can also include graphics reminding the healthcare worker that the sample collection tubes should be inverted after the blood sample is collected. The card 120 can also include a reminder that the sample collection tubes should be completely filled by the blood sample. The card 120 can also include a reminder of the proper order for filling the tubes with the blood sample and about the number of times each tube should be inverted after the sample collection tube is filled with the blood sample. For example, as shown in FIG. 11B, colored squares are arranged in a ring with a square corresponding to the first tube that should be filled positioned at twelve o'clock and a square corresponding to the last tube that should be filled position at about ten o'clock. The number of times that each tube should be inverted after the sample is obtained is written in the middle of each square. FIGS. 11C and 11D are photographs showing the instruction cards 120 of FIGS. 11A and 11B provided on containers 10 at appropriate locations.

Methods of Performing a Blood Draw and Collection Procedure with an Equipment Kit

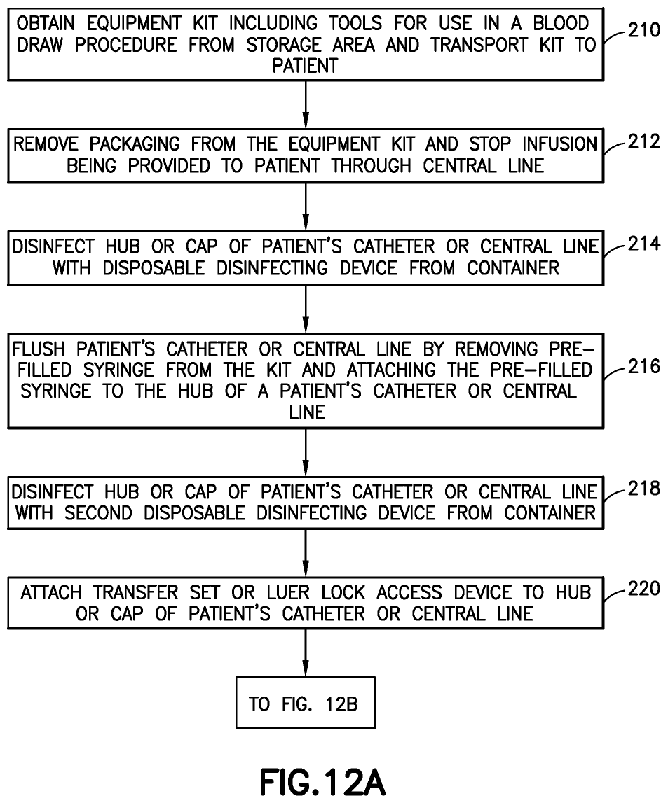

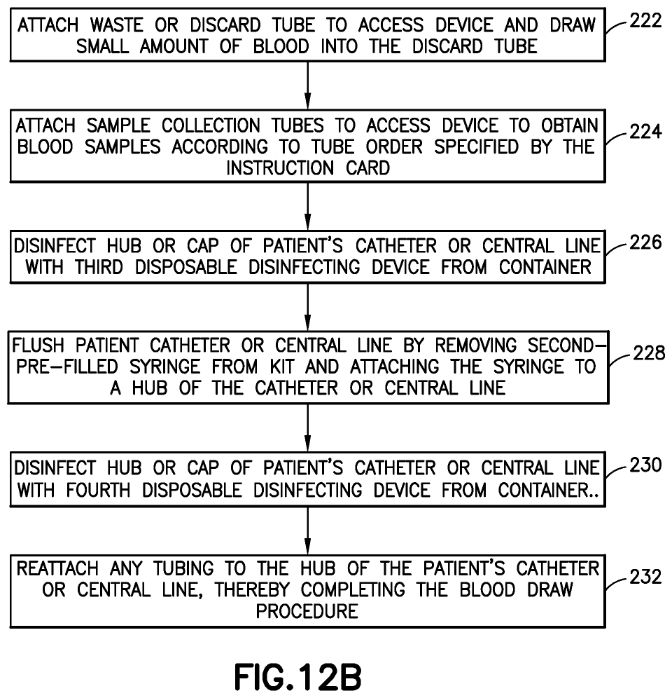

The equipment kits 110 and containers 10 of the present disclosure are used for performing blood draw and collection procedures and, in particular, can be used for performing a blood draw and collection procedure from a patient's catheter or central line. FIGS. 12A and 12B are flow charts showing steps for performing the blood draw and collection procedure using one or more of the previously described equipment kits 110 or containers 10.

As shown at step 210, the method includes obtaining an equipment kit 110 including a container 10 housing tools needed for performing the blood draw and collection procedure. The obtained equipment kit 110 can include features of any of the previously described kits 110 and containers 10, such as packaging 24 in the form of the folded cardboard cover 26 or the removable cover sheet 48, 50 adhered to the tray 12 of the container 10. As previously described, the equipment kit 110 can be stored at and retrieved from, for example, a medical facility storage area.

At step 212, the healthcare worker removes at least a portion of the packaging 24 from the container 10 of the equipment kit 110. Removing the packaging 24 can include, for example, swinging the top portion 30 of the folded cover 26 away from the tray 12 or removing the cover sheets 48, 50 adhered to the tray 12 from the tray 12. Once the packaging 24 is at least partially removed from the tray 12, at step 214, the healthcare worker disinfects the hub or cap of the patient's catheter or central line. For example, the healthcare worker may insert the first disposable disinfecting device 112 from the container 10 over the patient hub or cap. The healthcare worker may rotate or twist the disinfecting device 112 about the hub or cap to distribute disinfecting solution of the device 112 over surfaces of the hub or cap.

At step 216, the healthcare worker next removes one of the pre-filled syringes 116 containing flushing solution (i.e., saline, heparin, or another fluid solution) from the container 10 and attaches the syringe 116 containing the flushing solution to the hub of the patient's catheter or central line. The healthcare worker then expels the flushing solution from the syringe 116 through the patient's catheter or central line to flush the tubing to avoid contaminating the collected blood sample. After flushing, the syringe 116 can be disconnected from the hub and, at step 218, the healthcare worker again disinfects the patient's hub or cap using a second disposable disinfecting device 112 contained in the container 10.

At step 220, the healthcare worker next attaches a tube holder, such as a blood transfer set or tube holder 118, to the hub or cap of the patient's catheter or central line, thereby establishing a fluid access point for the central line through which the blood samples can be collected. At step 222, the healthcare worker next attaches the waste or discard tube 115 to the tube holder 118 and allows a small amount (i.e., about 5 mL to 10 mL) of blood to pass into the waste or discard tube 115 from the patient's catheter or central line. The waste or discard tube 115 and small amount of blood contained therein can be discarded, as the blood in the waste or discard tube 115 is not used for any blood test. Instead, this small amount of blood draw and collection into the discard tube 115 is considered to be waste because it may contain an amount the flushing solution.

At step 224, the healthcare worker next obtains the blood sample from the patient. Obtaining the blood sample can include attaching one of the sample collection tubes 114 to the transfer set or tube holder 118 and allowing blood to flow into the tube 114 through the transfer set or tube holder 118. Once the tube 114 is filled to an appropriate level with the blood sample, the healthcare worker can remove the filled tube 114 from the transfer set or tube holder 118 and can invert the filled tube 114 a number of times, as instructed on the instruction card 120. The healthcare worker can repeat the process of filling the sample collection tubes 114 in the order specified by the instruction card 120 until all required tubes 114 are filled. Alternatively, the blood sample can be drawn into a syringe 116. The healthcare worker can then provide the blood sample from the syringe 116 to each sample collection tube 114 using the transfer set or tube holder 118 or a similar connector, as are known in the art.

Once all required blood sample tubes 114 are filled with blood samples, the healthcare worker can remove the transfer set or tube holder 118 from the hub or cap of the patient's catheter or central line. At step 226, the healthcare worker then again disinfects the hub or cap by, for example, positioning a third disinfecting device 112 from the container 10 over the hub or cap and rotating or twisting the disinfecting device 112, as previously described.

Once the hub or cap has been disinfected, at step 228, the healthcare worker can again flush the patient's catheter or central line by attaching a second pre-filled syringe 116 retrieved from the container 10 to the hub or cap of the patient's catheter or central line and expelling the flushing solution through the catheter or central line to vasculature of the patient, as previously described.

At step 230, after flushing is completed, the healthcare worker removes the syringe 116 from the hub or cap and again disinfects the cap or hub using the fourth disinfecting device 112 in the container 10. Finally, at step 232, the healthcare worker can reattach any tubing (such as an IV drip or other medical tubing) to the hub of the patient's catheter or central line and restart the infusion through the tubing to the patient's vasculature, thereby completing the blood collection procedure.

While examples of the containers, equipment kits, and methods are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A container configured to store tools used by a healthcare worker when performing a blood draw and collection procedure, the container comprising:
a tray comprising a plurality of chambers sized to receive the tools used by the healthcare worker for the blood draw and collection procedure, the tools comprising at least one disposable disinfecting device for disinfecting portions of the tools and at least one tube for storing a blood sample; and
packaging that, in a closed or prior-to-use position, is over openings of the plurality of chambers of the tray, thereby securely containing the tools within the chambers of the tray and, in an open or ready-for-use position, is separated from the openings of the plurality of chambers of the tray so that the healthcare worker can access the tools contained within the chambers while performing the blood draw and collection procedure.

2. The container of claim 1, wherein at least one of the plurality of chambers is a waste chamber configured to receive the at least one disposable disinfecting device after use.

3. The container of claim 1, wherein the tray further comprises a plurality of tube holders sized to receive tubes to support the tubes in an upright position relative to the tray in an order in which the tubes are to be filled with the blood sample.

4. The container of claim 3, wherein the plurality of tube holders are formed from a plate over at least a portion of one of the plurality of chambers with circular openings having a diameter substantially matching a diameter of a cross-section of the tubes.

5. The container of claim 1, wherein the tray comprises a molded plastic tray.

6. The container of claim 1, wherein the packaging comprises a folded cover comprising a bottom portion, at least one side portion, and a top portion configured to swing between the closed position, in which the top portion of the cover is over the openings of the plurality of chambers of the tray, and the open position, in which the top cover is spaced apart from the openings of the plurality of chambers of the tray, so that tools and/or other items can be placed in or removed from the plurality of chambers of the tray.

7. The container of claim 6, wherein the top portion of the folded cover comprises a folded flap configured to be engaged to a slot, groove, flap, or hook on the at least one side of the folded cover, thereby securing the cover in the closed position.

8. The container of claim 6, further comprising one or more fasteners for mounting the tray to the bottom portion of the folded cover.

9. The container of claim 1, wherein the packaging comprises at least one removable cover sheet that is removably adhered to a top surface of the tray to secure the tools within the plurality of chambers of the tray.

10. The container of claim 9, wherein the at least one cover sheet comprises a first cover sheet over one or more of the plurality of chambers, and a second cover sheet over other chambers, and wherein the first cover sheet and the second cover sheet are separately removable from the tray.

11. The container of claim 10, further comprising a plate sized to fit within the opening of one of the plurality of chambers to prevent items from falling out of the chamber when the container is inverted.

12. The container of claim 1, wherein the blood draw and collection procedure comprises a procedure of collecting blood from a catheter of a patient.

13. The container of claim 12, wherein the tools further comprise one or more of: at least one syringe containing a solution, such as saline or heparin, for flushing the catheter of the patient; at least one of a blood sample transfer set or tube holder configured to be engaged to the at least one tube for filling the tube with the blood sample; or the disposable disinfecting device comprises a swab or brush comprising alcohol for disinfecting the tools and/or components of a vascular tube holder connected to the patient.

14. An equipment kit containing tools used by a healthcare worker when performing a blood draw and collection procedure, the kit comprising:
the container of claim 1,
the at least one disposable disinfecting device contained within at least one of the plurality of chambers of the tray; and
the at least one tube configured to contain the blood sample contained within another of the at least one plurality of chambers of the tray.

15. The kit of claim 14, further comprising at least one instruction card with instructions for one or more of preparing the tools for use prior to performing the blood draw and collection procedure, flushing fluid through a catheter of a patient prior to performing the blood draw and collection, wasting a sufficient amount of blood prior to performing the blood draw and collection, filling a plurality of the tubes with a blood sample in a correct order of tubes, and/or capping the catheter of the patient after drawing blood from the patient.

16. The kit of claim 14, wherein the blood draw and collection procedure comprises a procedure of collecting blood from a catheter of a patient, and at least one of a blood sample transfer set or tube holder configured to be engaged to the at least one tube for filling the tube with the blood sample.

17. The kit of claim 14, wherein the tools further comprise at least one syringe containing a solution, such as saline or heparin, for flushing a patient line.

18. The kit of claim 14, wherein the disposable disinfecting device comprises a swab or brush comprising alcohol for disinfecting components of the tools and/or of a vascular tube holder connected to the patient.

19. A method of performing a blood draw and collection procedure, comprising:
removing at least a portion of the packaging of the container of the kit of claim 14 from the tray so that tools contained in the tray can be removed from the tray;
disinfecting a hub or cap of a central line of a patient with the at least one disposable disinfecting device;
attaching a blood sample transfer set or tube holder contained in the container of the kit to the disinfected hub or cap such that the transfer set or tube holder is in fluid communication with the central line;
drawing a blood sample from the central line of the patient through the transfer set or tube holder into the at least one tube, thereby providing the blood sample obtained from the central line of the patient; and
detaching the blood sample transfer set or tube holder from the hub or cap.

20. The method of claim 19, wherein the kit further comprises at least two syringes, each containing a solution, such as saline or heparin, for flushing the central line of the patient, the method further comprising flushing the central line of the patient with the syringes containing the flushing solution both before attaching the blood sample transfer set or tube holder to the hub or cap and after detaching the blood sample transfer set or tube holder from the hub or cap.
